(19) United States
(12) Patent Application Publication
(10) Pub. No.: US 2026/0266784 A1
(43) Pub. Date: Sep. 10, 2026

(54) SYSTEM AND METHOD FOR THIN LAYER CHROMATOGRAPHY

(71) Applicant: SAFETY FIRST TESTING SOLUTIONS, INC., San Francisco, CA (US)

(72) Inventors: George FARQUAR, San Francisco, CA (US); Marcus TOFANELLI, San Francisco, CA (US); Katrina Marie MYERS, San Francisco, CA (US)

(21) Appl. No.: 19/165,550

(22) PCT Filed: Mar. 15, 2024
(86) PCT No.: PCT/US24/20322

§ 371 Date: Sep. 15, 2025

Related U.S. Application Data
(63) Continuation of application No. PCT/US2023/015329, filed on Mar. 15, 2023.
(60) Provisional application No. 63/582,501, filed on Sep. 13, 2023.

(51) Int. Cl. G01N 30/91; B01D 15/18; B01D 15/22; G01N 30/94; G01N 33/487

(52) U.S. Cl. CPC G01N 30/91; B01D 15/1878; B01D 15/222; G01N 30/94; G01N 33/487

(57) ABSTRACT

Provided herein are systems, articles and methods for automatically performing thin layer chromatography. The methods comprise automatically extracting an analyte from a sample in a cartridge comprising a fluidic chip and phase transfer assembly, automatically depositing the analyte on a thin-layer chromatography plate and developing the plate, and automatically detecting the analyte on the developed plate.

### REFERENCE TO RELATED APPLICATIONS

This application claims the benefit of the priority date of International Patent Application No. PCT/US23/15329, filed on Mar. 15, 2023, and U.S. Provisional Application No. 63/582,501, filed Sep. 13, 2023, the contents of which are incorporated herein by reference in their entireties.

### STATEMENT AS TO FEDERALLY SPONSORED RESEARCH

None.

### THE NAMES OF THE PARTIES TO A JOINT RESEARCH AGREEMENT

None.

### SEQUENCE LISTING

None.

### BACKGROUND

Thin layer chromatography (“TLC”) is an analytical tool that separates analytes based on differential migration speeds through a thin layer of chromatographic medium. Analyte migrates in the direction of a separation solvent moving through the medium by capillary action.

TLC has been used to detect drug analytes in human bodily fluids. For example, Meuller et al. used TLC to detect benzoylecgonine in human urine. (Meuller et al., “Detection of benzoylecgonine in human urine,” Journal of Chromatography A, Volume 144, Issue 1, 1977, Pages 101-107.) See also U.S. Pat. No. 10,458,963, Oct. 29, 2019 (“Quantitative HPTLC cannabinoid field testing device and method”).

Detection of analytes by TLC poses challenges, including exchange of and/or transfer of one or more analytes from a first liquid to a second liquid, fractionation of certain samples to remove contaminants before application to the TLC plate, and automation of the method to start with a raw sample and produce a chromatographic analysis.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. The invention will be more particularly described in conjunction with the following drawings wherein:

FIGS. 1A-1E shows an exemplary chromatography cartridge. The cartridge has dimensions of about 11.5 cm×11.5 cm. FIG. 1A shows the fluidic circuitry. FIGS. 1B and 1C show top and bottom and views, respectively, of the cartridge. FIGS. 1D and 1E show two aspects of a cartridge comprising a phase transfer assembly.

FIG. 2 shows an exemplary chromatography plate seated in a cartridge support.

FIGS. 3A and 3B show an exemplary reservoir formed from the mating of a fluidic chip and a TLC plate.

FIG. 4 shows an exemplary layout of reservoirs on a chromatography plate.

FIGS. 5A and 5B show exemplary phase transfer assemblies.

FIG. 6 shows a diagram of an exemplary fluidics assembly.

FIG. 7 shows aspects of an exemplary fluidic manifold.

FIG. 8 shows an exemplary cartridge interface.

FIG. 9 shows elements of an exemplary system.

FIG. 10 shows an exemplary Peltier device position under cartridge stage.

FIGS. 11A-11C show an exemplary ball valve in a fluidic chip. FIG. 11A shows a fluidic chip with a ball valve seated in a fluidic channel. FIG. 11B shows the valve in closed position with the ball seated in an O-ring, closing the channel. FIG. 11C shows the valve in open position with the ball pushed out of the O-ring.

FIG. 12 shows an exemplary fluidic chip 100 comprising a sample container 1010 engaged with port 101 of the chip. Further shown is phase transfer assembly 130 positioned external to the fluidic chip.

FIG. 13 shows an exemplary syringe assembly 1304 comprising a syringe 1301 comprising a notch 1302 and a metering clip 1303, the syringe assembly configured to limit the amount of sample loaded into the syringe by a user.

### SUMMARY

In one aspect, provided herein is a system for automatically performing thin layer chromatography. The system automatically performs and integrates the following operations: (1) extraction of analytes from a sample and delivery of the analytes to a loading area of a thin layer chromatography plate; (2) development of a thin layer chromatography plate to separate analytes from other molecules in the sample; and (3) detection of analytes and analysis of results. The system also automatically delivers to the chromatography plate separation medium to separate analytes in the sample by capillary action. The system also automatically compresses a sample band by delivering concentration medium to the chromatography plate to one or both sides of the sample band. The system also automatically delivers detection agents to the chromatography plate to allow detection, such as visualization, and measuring of the analyte on the plate. The system also automatically detects the analyte with the detector, such as a camera. The system further comprises a computer that measures the analyte on the plate from the signal or image provided by the detector. The system also can comprise auxiliary elements such as fans and heaters to evaporate liquids from the chromatography plate.

Additionally or alternatively, systems provided herein automatically performs and integrates the following operations: (1) exchange and/or transfer of one or more analyte molecules from a liquid sample into a second liquid, optional extraction of analytes from the liquid sample, and delivery of the analytes to a loading area of a thin layer chromatography plate; (2) development of a thin layer chromatography plate to separate analytes from other molecules in the sample; and (3) detection of analytes and analysis of results.

In certain embodiments, the system comprises an instrument configured to engage a cartridge. The cartridge includes a thin layer chromatography plate and a fluidic chip comprising fluidic circuits including fluidic channels and ports that engage a sample container and ports in a fluidic assembly of the instrument. The instrument further comprises pumps and valves that, when operated, move a sample fluid from the sample container into the fluidic chip, and from the fluidic chip onto the chromatography plate. Other pumps and valves in the instrument move liquids from liquid reservoirs into fluid channels in the fluidic chip and out of the fluidic chip onto the chromatography plate. The cartridge can also comprise phase transfer media that allows separation of contaminants from the analyte, and allows analyte to pass through to the chromatography plate. The chromatography plate and the fluidic chip are made in the cartridge such that a combination of apertures in the fluidic chip and a surface of the chromatography plate form barriers creating wells or reservoirs. Liquid deposited in the wells is contained by the wells but can migrate through the chromatographic material under the barriers. The chromatographic chip also comprises one or more apertures, optionally covered with a transparent material, that expose a detection area of the chromatographic plate, allowing analyte in the detection area to be visualized.

Methods described herein allow the detection of analytes, such as cannabinoids, and, in particular, THC, in a sample, such as saliva, at significantly low levels. For example, methods as described herein can detect as little as any of 50 ng, 20 ng, 5 ng, 2 ng, 1 ng or 0.2 ng in a saliva sample of about 400 microliters. That is, the methods allow detection of cannabinoid such as THC at concentrations as low as any of 125 nanograms per milliliter, 50 nanograms per milliliter, 12.5 nanograms per milliliter, 2.5 nanograms per milliliter or 0.5 nanograms per milliliter.

Furthermore, the detection process can be performed quickly. For example, the process of loading the sample on the cartridge, extracting the analyte, and loading the analyte onto the chromatography plate, can be performed in no more than any of 30 minutes, 10 minutes, 5 minutes, 1 minute or 30 seconds. The entire time from loading the sample on the cartridge to extracting the analyte, loading the analyte onto the chromatography plate, developing the plate, and detecting the analyte, can be performed in no more than any of 1 hour, 45 minutes, 30 minutes, 15 minutes, or 5 minutes.

### DETAILED DESCRIPTION

### I. Introduction

Disclosed herein are articles, systems, and methods for quickly testing for and precisely quantifying a dilute analyte in a sample, also referred to herein as a mixture, e.g., a drug in a biological sample. The articles, systems, and methods described herein can be performed in any setting with the required materials and can provide quantitative results for a variety of different compounds or substances of interest. The system can be configured to be used in the field for in situ testing, or in an office or laboratory setting.

The sample can comprise one or more analytes of interest. In certain embodiments, the sample can be, for example a biological sample, a water sample, a food sample, an agricultural sample, an industrial sample, a research sample, or any other suitable sample tentatively comprising an analyte of interest. Typically, the sample is a liquid sample, such as an aqueous liquid sample comprising an aqueous solvent and tentatively one or more analytes.

The articles, systems, and methods disclosed herein can quickly, e.g., in 20 minutes or less, provide accurate and precise quantitative analysis of one or more analyte. The analyte can be present in dilute concentrations in the sample, and the apparatus, systems, and methods configured to enable quantification of the dilute analytes. The articles, systems, and methods allow the analysis of a volume, e.g., at most about any of 5 mL, 4 mL, 3 mL, 2 mL, 1 mL, 0.5 mL, 0.25 mL, or 0.1 mL, of a sample comprising an analyte, for example at most about 2 mL of sample comprising an analyte.

Methods disclosed herein can comprise any suitable method for performing separation and/or concentration of at least one analyte in a sample. Methods disclosed herein can comprise any suitable method for detecting at least one separated and/or concentrated analyte. Methods disclosed herein can comprise any suitable method for applying an indicator, e.g., a dye, to the chromatography medium. As used herein, the terms “indicator” and/or “dye” include any compound that changes a property of an analyte. Typically, the indicators and/or dyes change the properties of the analyte in such a way as to enable and/or improve detection of the analyte, for example, by attaching a detectable label. Methods disclosed herein can comprise any suitable method for quantifying at least one separated and/or concentrated analyte.

Articles disclosed herein can comprise a flat, solid substrate coated at least in part with chromatography medium. The article can contain any number of fluidic barriers and/or gates to direct the flow of solvent, and as a result, direct the flow of analytes and non-analytes in the sample across the chromatography medium. The article can comprise one or more reference standards to aid in quantification of the one or more analyte.

Systems disclosed herein can comprise a receiving area for disclosed articles, at least one solvent chamber configured such that the at least one solvent chamber is in fluid communication with at least a portion of the chromatography medium, and/or a detector. The system and the article can be configured such that the article is in the form of a replaceable cartridge and the system is configured in such a way to receive the replaceable cartridge. The system can be configured such that the cartridge is oriented horizontally or vertically.

### II. Cartridge

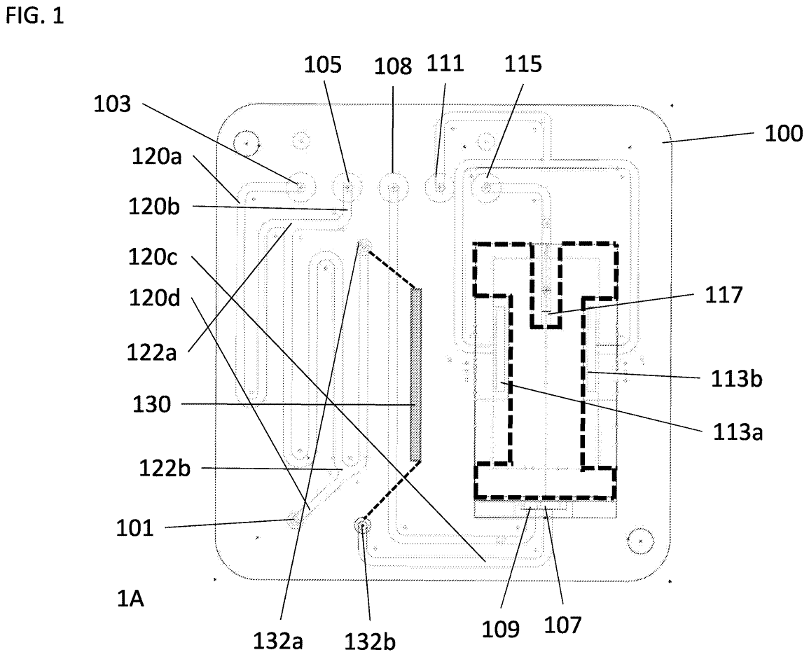

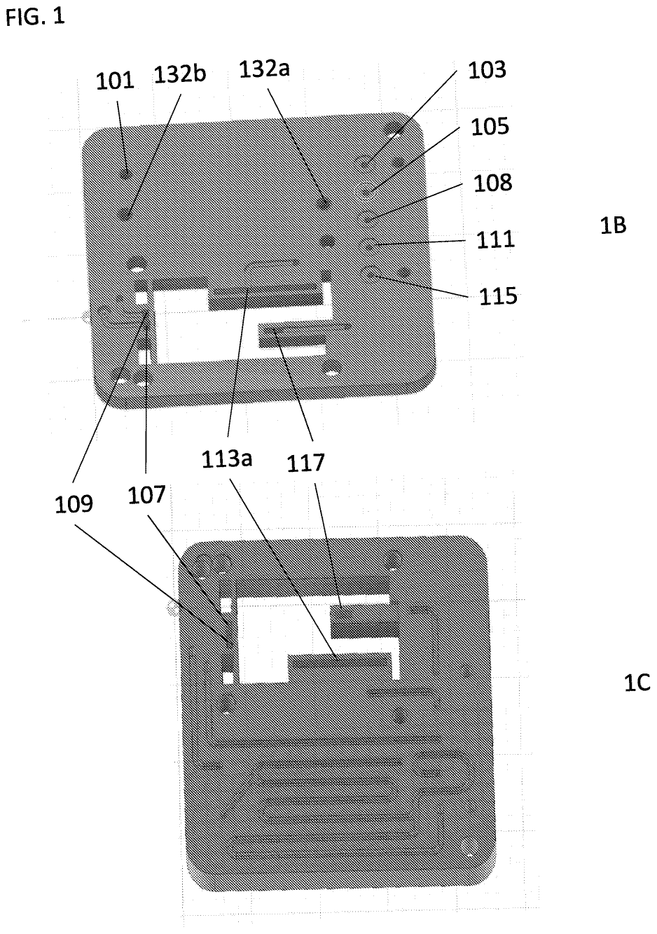

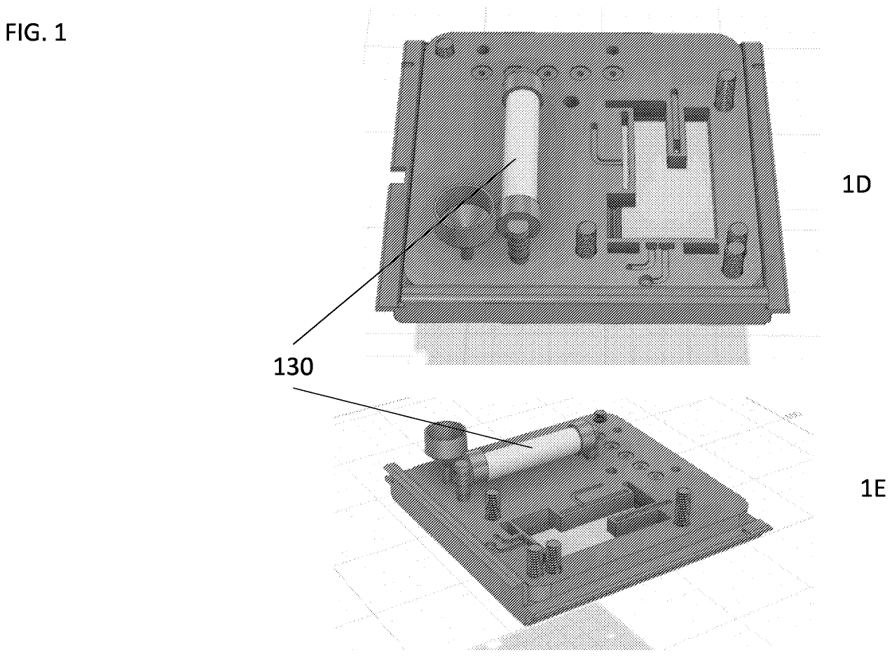

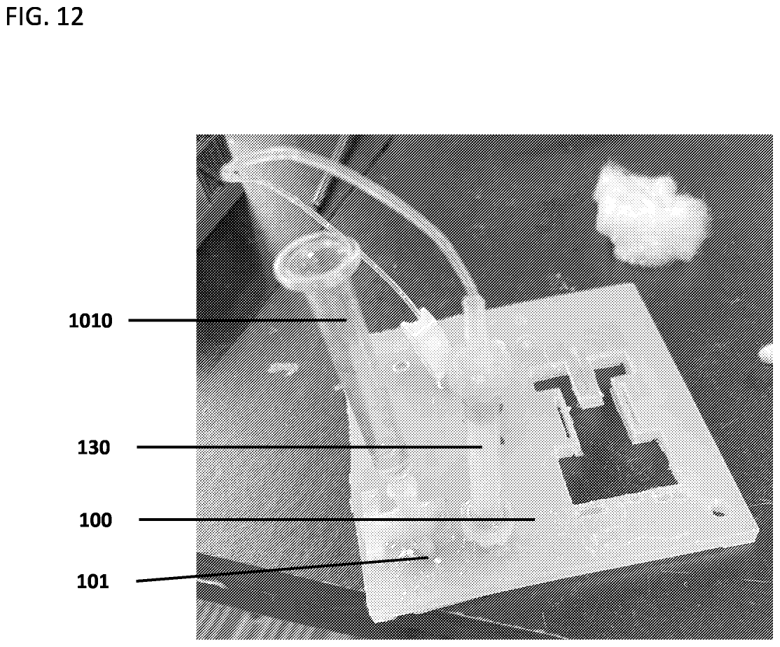

Cartridges of this disclosure are configured to receive sample and various solutions into fluidic channels of the cartridge, and deliver them to various locations on a chromatography plate for performing thin layer chromatography (“TLC”). Accordingly, such cartridges comprise a TLC plate comprising a surface comprising chromatographic medium and, mated thereto, a fluidic chip comprising fluidic circuits including fluidic channels communicating with ports. The cartridge further can comprise a phase transfer assembly. The phase transfer assembly can comprise a channel and/or column comprising phase transfer media. The phase transfer assembly can be positioned external to or internal to the fluidic chip. The long axis of the phase transfer assembly can be positioned at any suitable angle with respect to the plane of the cartridge, such as parallel to (for example, as shown in FIG. 1) or normal to the plane of the cartridge (for example, as shown in FIG. 12). The phase transfer medium separates analytes in the sample from other materials so that a more purified sample can be delivered to the chromatography plate.

### A. TLC Plate

TLC plate can comprise a solid substrate coated with a chromatographic medium.

### 1. Substrates

The substrate can comprise any suitable material, for example, glass, quartz, metal, aluminum, plastic, or a suitable alternative. The substrate can comprise any suitable shape. The substrate can be triangular, rectangular, square, trapezoidal, rhomboidal, pentagonal, hexagonal, heptagonal, octagonal, circular, elliptical, or the like. The substrate can comprise a thickness of at least about any of 0.1 mm, 0.25 mm, 0.5 mm, 0.75 mm, 1 mm, 2 mm, 3 mm, 4 mm, 5 mm, 6 mm, 7 mm, 8 mm, 9 mm, or 10 and/or no more than about any of 0.1 mm, 0.25 mm, 0.5 mm, 0.75 mm, 1 mm, 2 mm, 3 mm, 4 mm, 5 mm, 6 mm, 7 mm, 8 mm, 9 mm, 10 mm, or 20 mm, for example, the substrate can comprise a thickness of about 0.1 to about 20 mm, preferably, the substrate can comprise a thickness of about 0.1 to about 5 mm, about 0.1 um to about 2 mm, or about 0.1 um to about 1 mm. The substrate can have a thickness between 1 mm and 2 mm. The substrate can comprise 3 geometric axes, a first axis comprising the thickness of the substrate (herein referred to as the z-axis) and two additional, coplanar axes typically about perpendicular to the first axis. The second axis refers to the length of the substrate (herein termed the “x-axis” or the “first dimension” of the substrate), and the third axis refers to the width of the substrate (herein termed the “y-axis” or the “second dimension” of the substrate). The first and the second dimensions of the substrate can be at any angle relative to each other. The second dimension is at an angle between about 45 degrees and about 135 degrees, between about 75 degrees and about 105 degrees, between about 80 degrees and about 100 degrees, about 85 degrees and about 95 degrees, or about 90 degrees of the first dimension. The second dimension is about orthogonal to the first dimension. In certain embodiments the second dimension is substantially orthogonal to the first dimension. The substrate can comprise a first dimension and/or a second dimension of at most about any of 50 cm, 45 cm, 40 cm, 35 cm, 30 cm, 25 cm, 20 cm, 15 cm, 10 cm, 5 cm, 4 cm, 3 cm, 2 cm, 1 cm, or 0.5 cm and/or at least about any of 45 cm, 40 cm, 35 cm, 30 cm, 25 cm, 20 cm, 15 cm, 10 cm, 5 cm, 4 cm, 3 cm, 2 cm, 1 cm, 0.5 cm, or 0.25 cm long, for example, about 50 to about 0.25 cm long, preferably about 30 to about 5 cm long, more preferably about 30 to about 20 cm long, yet more preferably at most about 25 cm long. The length of the first dimension and the length of the second dimension are not the same. The length of the first dimension and the length of the second dimension are the same.

### 2. Chromatography Medium

The substrate is at least partially coated with a chromatography medium. The top surface of the substrate is at least about any of 70%, 80%, 90%, 95%, 96%, 97%, 98%, 99%, or 99.5% and/or no more than about any of 80%, 90%, 95%, 96%, 97%, 98%, 99%, 99.5%, or 100% coated with chromatography medium, for example about 70% to about 100% coated with chromatography medium, preferably about 85% to about 100% coated with chromatography medium. The chromatography medium can comprise any suitable medium as recognized by a skilled artisan. The chromatography medium can comprise silica, alumina, cellulose, polyamide, or the like. In certain instances, the chromatography medium can comprise one or more chemical modifications such as C2, C8, C10, C18, phenol, amine, and/or chiral. The chromatography medium can be functionalized with any suitable chemical modification. The chromatography medium can be functionalized with C2, C8, C18, or any other suitable alternative as recognized by a skilled artisan. The chromatography medium can comprise a fluorescent molecule whereby the chromatography medium fluorescence upon exposure to electromagnetic radiation, for example compounds which fluoresce upon exposure to short wavelength UV, e.g., 254 nm, such as tin-activated strontium compounds, uranyl acetate, magnesium-activated zinc silicate, zinc cadmium sulfide, and the like. The chromatography medium can comprise an average particle size ranging from 1 um to 50 um. The chromatography medium can comprise any suitable thickness for the application. The thickness of the chromatography medium can comprise at most about any of 500 um, 400 um, 300 um, 200 um, 100 um, 75 um, 50 um, or 25 um and/or at least about any of 400 um, 300 um, 200 um, 100 um, 75 um, 50 um, 25 um, or 10 um, for example the thickness of the chromatography medium can comprise about 500 um to about 10 um.

The chromatography medium is attached to the substrate using a binder. The binder can be any suitable binder, for example an organic binder, an inorganic binder, and/or gypsum.

### 3. Substrates Comprising Calibrants

In certain embodiments, the article comprises a calibrant. As used herein, a “calibrant” includes any molecule identical or similar to an analyte that is applied at one or more amounts onto the chromatography medium. The calibrants can provide an expected signal upon detection. In certain embodiments, the one or more calibrants can be fluidically isolated from the rest of the plate. In other embodiments, the one or more calibrants can be adjacent to but not in contact with the sample, wherein the calibrant travels through the chromatography medium analogous to the one or more analytes in the sample. In certain embodiments, the calibrant and analyte are fluidically isolated during analysis. In certain embodiments, one or more calibrants are added to the chromatography medium during manufacturing, before use, during use, or after use. In certain embodiments, the calibrants are positioned within a gate such that separating and/or concentrating solvent are not able to disturb the calibrants during separation and/or concentration steps. The calibrants can be placed in any suitable position on the chromatography medium. It is typically preferred that the calibrants are placed outside of the path of travel of any component of the sample during the separation and/or concentration steps in order to prevent disruption of the solvent flow path. In certain embodiments, the calibrants are placed in an area fluidically isolated from the rest of the chromatography medium.

Any suitable number of calibrants can be placed on the substrate, such as no more than any of 10, 9, 8, 7, 6, 5, 4, 3, 2, or 1 different concentrations of an analyte to be quantified.

### B. Fluidic Plate

### 1. Plate Construction

The fluidic plate can comprise one or more networks of internal channels that open on ports. Plate can be formed of a single piece into which fluidic channels are introduced, e.g., by laser etching. More typically, the plate can comprise two pieces are mated with each other. One or both of the pieces can be etched to introduce grooves or troughs onto the surface of the piece. Mating the two pieces covers and closes the channels so that they are internal to the fluidic plate. One or both pieces also comprise holes, e.g., apertures or vias, so that, for example, a first side of the plate communicates with the second side of the plate through the aperture. Apertures can be positioned to communicate with the fluidic channels. For example, an aperture can be positioned at the end of a channel. Apertures can be included on the piece comprising the grooves, or can be positioned on the opposing piece and aligned with a group on the opposite piece.

The pieces can be constructed of any material known to those skilled in the art. For example, the cartridge can be constructed of a plastic, glass, or metal. A plastic material may include any plastic known to those skilled in the art, such as polypropylene, polystyrene, polyethylene, polyethylene terephthalate, polyester, polyamide, poly(vinylchloride), polycarbonate, polyurethane, polyvinyldiene chloride, cyclic olefin copolymer, or any combination thereof. The fluidic plate can be formed using any technique known to those skilled in the art, such as soft-lithography, hard-lithography, milling, embossing, ablating, drilling, etching, injection molding, or any combination thereof.

### 2. Fluidic Circuits

The fluidic plate can comprise one or more fluidic circuits. Fluidic circuits comprise ports in the fluidic plate connected by one or more internal and, optionally, external, fluidic channels.

Referring to FIGS. 1A-C, fluidic chip 100 can comprise a first fluidic circuit comprising a sample inlet port 101, a first pressure port 103, a second pressure port 105 and a fluid delivery port 108, all connected through fluidic channels 120a-d. Port 103 and fluidic channel 120a are optional aiding with sample positioning from a sample collector in certain cases. A port may communicate with two or more other ports through junctions or intersections 122a-b between fluidic channels that form branches in the circuit.

Referring to FIG. 12, a sample container 1010 can engage with sample inlet port 101 to provide sample fluid to the fluidic chip.

The fluidic plate also can comprise a second fluidic circuit including a separating solvent intake port 108 and a separating solvent delivery port 109. The separating solvent delivery port is positioned to deliver separating solvent onto a sample loading zone of the chromatographic plate. In some embodiments, the second fluidic circuit can share ports and/or fluidic channels with the first fluidic circuit.

The fluidic plate also can comprise one or more third fluidic circuits. The circuits can comprise one or more concentrating solvent intake ports 111 and one or more concentrating solvent delivery ports 113a and 113b, positioned to deliver concentrating solvent to the one or more concentrating solvent loading zones.

The fluidic plate can also comprise one or more fourth fluidic circuits comprising ports connected by one or more fluidic channels, including a dye intake port 115 and a dye delivery port 117, wherein the dye delivery port is positioned it to delivery dye onto a separation zone of the chromatography plate. Additionally or alternatively, the cartridge may comprise dye in the port 115 and/or the fluidic conduit between port 115 and 117, for example dried dye forming a dye film on the surface of the port or fluidic conduit. Dye may be added to the cartridge during manufacturing using any suitable method, for example introducing solvated dye into port 115 and/or the fluidic conduit between port 115 and 117, wherein the dye is dried to form a film in the port or the fluidic conduit. In certain cases, the addition of dye to the cartridge during manufacturing may be beneficial to increase the shelf life of the dye as certain dyes begin to decompose after solvation. Dried dye can be resuspended by introducing an appropriate solvent from a solvent reservoir by the pumping system, allowing the dye to solvate in the solvent, and optionally mixing the dye to homogeneity. An exemplary method to mix the dye to homogeneity is to draw the solution comprising the solvated dye into the pumping system one or more times before applying the homogeneous solution into port 115, through the fluidic conduit, and out port 117 to the TLC plate for use.

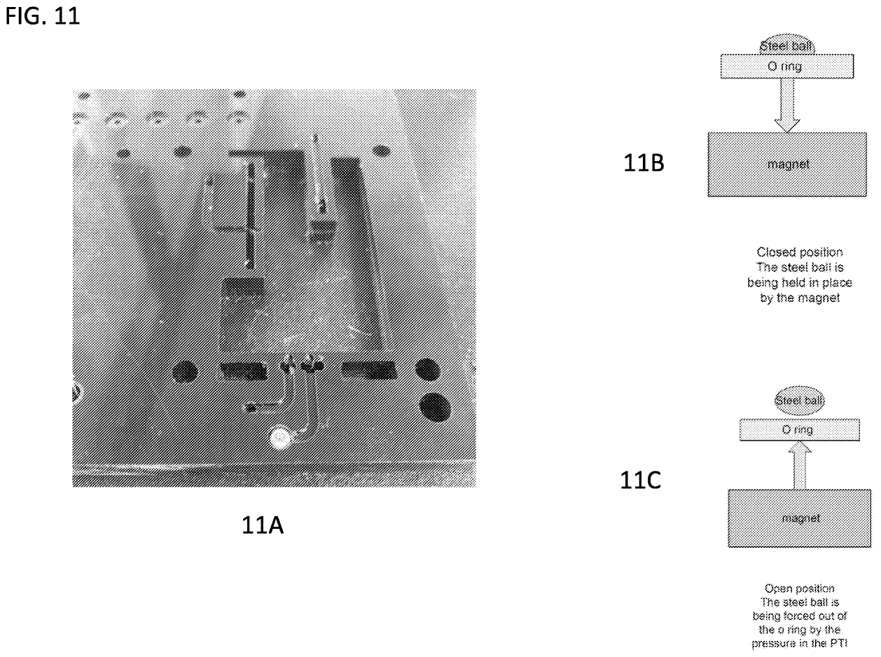

The fluidic plate also can comprise a valve positioned between the phase transfer assembly and the sample loading zone. The valve can be, for example, a ball valve seated in an O ring and held in place by a magnet. (See FIGS. 11A-C.) When vacuum is pulled to introduce sample, the valve prevents air from being introduced from the sample loading zone. However, positive pressure to move sample through the phase transfer assembly displaces the ball, allowing passage of fluid into the sample chamber. Any suitable valve can be used. These include, without limitation, check valves, diaphragm valves, Quake valves, pinch valves, and electromagnetic valves.

### C. Sample Preparation and Phase Transfer Assembly

The first fluidic circuit can further include a phase transfer assembly. The phase transfer assembly can comprise a fluidic channel and/or column comprising filtration and chromatographic (“phase transfer”) media that is positioned downstream of the sample inlet port, and the two pressure ports, and upstream of the sample delivery port. In one embodiment the phase transfer assembly comprises a fluidic channel internal to the fluidic chip, the fluidic channel comprising the phase transfer media. In an alternative embodiment, depicted in FIGS. 1D and 1E, the phase transfer assembly comprises a column 130 comprising phase transfer media is contained in a housing 621a external to the fluidic chip and communicates with the first fluidic circuit through phase transfer connection ports 132a and 132b, e.g., through tubing. The long axis of the phase transfer assembly can be positioned at any suitable angle with respect to the plane of the cartridge, such as parallel to (for example, as shown in FIG. 1) or normal to (for example, as shown in FIG. 12) the plane of the cartridge. In certain cases, positioning the long axis of the phase transfer assembly normal to the plane of the cartridge may be advantageous and provide a lower limit of detection as compared to a parallel placement.

A phase transfer assembly performs three primary functions. First, it serves to filter out particulate matter in the sample. In the case of saliva, this includes, for example, food particles. Second, it serves to transfer and/or exchange one or more analytes in a liquid sample, such as an aqueous sample comprising an aqueous solvent, e.g., water, to an organic solvent for thin layer chromatography, thereby transferring the one or more analytes within the liquid sample to the organic solvent. Third, it serves as a stationary phase chromatographic medium to selectively bind and elute molecules, e.g., analytes, using different mobile phases. Any suitable mobile phase can be used, such as an aqueous phase, e.g., an aqueous solvent, and/or an organic phase, e.g., an organic solvent. In certain cases, the stationary phase chromatographic medium selectively binds to one or more undesirable molecules in the sample, e.g., non-analytes, thereby removing them from downstream analysis. The removal of the one or more undesirable molecules may improve the performance, e.g., limit of detection, sensitivity, resolution, and the like, of the downstream analysis of one or more analytes. Additionally or alternatively, the stationary phase chromatographic medium selectively binds to one or more desirable molecules in the sample, e.g., analytes, which are subsequently eluted for downstream analysis.

These three functions are performed typically by three material layers in the phase transfer assembly—a filter layer, a selective binding layer and a phase transfer layer. Each layer can contribute to more than one function.

Solvents that can be used include, for example, methanol, hexanes, water, acetonitrile, dichloromethane, dibromomethane, chloroform, toluene, acetone, methyl-thf, pentane, cyclopentyl methyl-ether, ethyl acetate, and 1-octanol. The selection of concentrating or separating solvent can depend on the stationary phase and the analyte. For THC detection, dichloromethane can be used to load and concentrate the analyte. Dibromomethane can be used as a separating solvent for THC as an analyte. Any organic analyte can be concentrated and separated with the correct mixture of solvents. Trinitrotoluene (a high explosive) and malathion (an organic pesticide) have been demonstrated to be detectable using this technology.

In this configuration, water from a saliva sample will be trapped on the phase transfer media within the phase transfer assembly, and analyte dissolved in an organic solvent pass through to be deposited on the plate.

For example, the phase transfer assembly can include a housing having layered media, e.g., beads therein. The phase transfer assembly can comprise a filter layer to remove solid particles from the sample. It can also comprise a hydrophilic adsorbent to absorb water. It also can comprise a sorbent for solid phase extraction of the analyte, e.g., THC. Isolation of one or more compounds of interest can proceed by first loading the sample into the phase transfer assembly and then flowing a solvent through the phase transfer assembly from the proximal end to the distal end. The fluid can thus move through the filter, then through the media, e.g., beads, of the phase transfer layer, and finally through the sorbent at layer (e.g., via force from the pumping system). The resulting extracted compound that exits at the distal end can then be sent to the detector for analysis.

### 1. First Screen Layer

A first screen 610a holds the layers in place in the housing. Any suitable screen can be used, such as a mesh, for example an aluminum screen.

### 2. Filter Layer

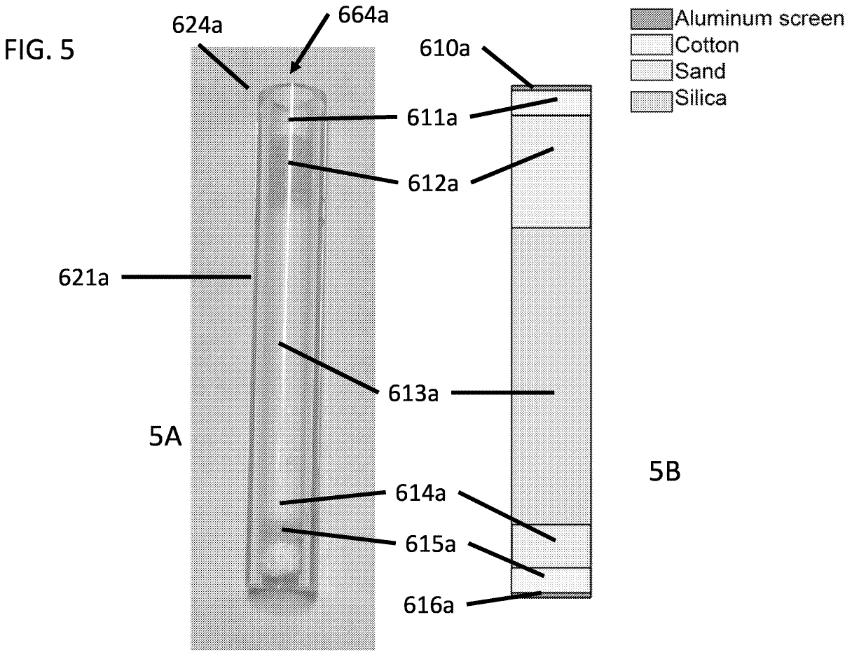

Referring to FIGS. 5A and 5B, the filter layer 611a can be used for samples that are heterogeneous mixtures, such as biological fluids, colloids, emulsions, or powdered/porous solids, to prevent clogging of the phase transfer interface 664a. A layer of fibrous material, in this case cotton, further serves to capture particles. Cotton has a variable pore size and prevents passage of particles in range of size from 20-100 μm. Alternative materials include glass wool, filter paper, or cloth. Cotton also can absorb water.

In some embodiments, the filter layer 611a can include beads that are made of one or more inert materials, such as polystyrene, sand or quartz sand. In other embodiments, the beads in filter layer 611a can be composed of chemically active materials designed to pretreat the samples for the rest of the cartridge/analysis (e.g., beads with C-18 coating, chiral coatings, derivatizing or chemical modification agents, or biological active compounds such as immune assays). The overall pore size of the filter layer 611a can be controlled by the size of the beads used, which can be spherical, shaped, or irregular, as long as they exhibit a narrow dispersion of sizes. Beads of the filter layer 611a can be 40-600 microns in diameter with a disparity of 20% or less. In some embodiments, the beads of filter layer 611a can be 40-60 microns in diameter. In other embodiments, the particles can be 300-600 microns.

In certain embodiments, the phase transfer layer comprises a single filter layer 611 that accommodates any biological sample without prior preparation (e.g., because the filter can capture large particles in the open pores, capture interferants by sticking them to the large surface area, and/or have internal pores that collect compounds).

The pore size and subsequent choice of filter layers depends on the sample to be extracted. For a homogenous sample, no filter layer is required, however for heterogenous (i.e., colloid, or emulsion) samples one or more layers can be employed. For example, many biological samples contain both colloidal and emulsified components with widely varying sizes. Thus, employing a single filter would either provide poor filtration or become clogged. The choice of the material depends on the desired extracting solvent analyte, as some filters could be chemically incompatible with either, or bind the analyte.

The filter layer can comprise media that filters out particles in sample having a size greater than any of 0.001 micron, 0.01 micron, 0.1 micron, 1 micron, 10 microns, or 100 microns.

### 3. First Selective Binding Layer

In some embodiments, the selective binding layer 612a can include material that binds chemicals or proteins that may interfere with analysis and/or prevent purification. For example, where thin layer chromatography is used as part of the detector, water can interfere with analysis. In such a case, particles of the binding layer 612a can be formed of desiccant materials.

A layer of hydrophilic particles, such as sand, 612A, functions both to capture particles and to adsorb water. For example, the layer can comprise sand of white quartz with particle sizes ranging from 200-300 μm. The pore size is 10-30 μm. Alternative materials include silica spheres or glass beads.

In other embodiments, silica or molecular sieves can be used to capture the water (and therefore remove it from the extracted compound). As another example, where the collected fluid comprises biological material, biological matrices can prevent low limits of detection for a variety of analyses. In such a case, beads with functional groups such as such as, thiols, COOH, NH₃ or CHO can be used for the binding layer 612a to remove the biological matrix from the fluid (and thus from the final extracted compound). In other examples, beads of layer 612a can be functionalized to bind specific chemical groups or destroy compounds. In some embodiments, the selective binding layer 612a can be combined with the phase transfer layer 613a.

### 4. Phase Transfer Layer

The phase transfer layer 613a can include phase transfer media, such as chromatographic media and/or sorbent media, e.g., particles or beads, that reversibly bind compounds, through physical or chemical interactions with different affinities depending on the choice of stationary phase and mobile phase. Examples of phase transfer media include quartz, alumina, polystyrene, silica, and beads functionalized with moieties such as C18, NH₃, or COOH.

Phase transfer media serves as a stationary phase. The phase transfer media selectively binds and elutes analytes depending on the solvent used. In the case of THC, and other cannabinoids, a normal phase material, such as silica, alumina, diatomaceous earth, and magnesium silicate (e.g., florisel) can be used. Silica has irregular particles. They can range in size from, e.g., 40-63 μm and a pore size of about 60 Angstrom. When an intermediate polarity solvent, such as dichloromethane (DCM), is used, Delta-9-tetrahydrocannabinol (Δ9-THC, or THC) and cannabidiol (CBD) are eluted from the silica, while metabolites (such as 11-Hydroxy-Δ⁹-tetrahydrocannabinol (11-OH-THC) and 11-Nor-9-carboxy-Δ⁹-tetrahydrocannabinol (11-COOH-THC) remain bound.

A compound's affinity largely depends on the polarity of the solvent, thus by changing the solvent from polar (methanol) to non-polar (hexanes) allows for multiple different sets of compounds to be obtained or isolated (and this can be done several times). For example, THC can bind to a reverse phase sorbent when the mobile phase is a non-polar solvent, such as water. However, when the mobile phase becomes non-polar, such as, dichloromethane (DCM), the analyte is released from the sorbent and with the mobile phase. For example, flowing hexanes over a sample can release non-polar compounds (e.g., THC) from the phase transfer interface while the polar compounds are left behind. Alternately or additionally, the polar compounds (e.g., THCA) can collected by flowing a polar solvent, such as methanol through the phase transfer assembly. Furthermore, the compound's affinity for both solvent and phase transfer media can be changed to protonate or deprotonate the compound, which can be controlled through the solvent's pH or inherent acid base nature of the solvent, for example triethyl amine. This allows the compound of interest to be collected while removing impurities. Furthermore, the amount of solvent required to remove a compound also varies with a compound's affinity for the phase transfer media, e.g., beads. Compounds of interest can be further targeted by changing the affinity of a compounds for the phase transfer media's surface. Furthermore, phase transfer media can be utilized to control the pH and act as a buffer, which can also protonate or deprotonate a compound, thereby affecting a compound's affinity for solvent and media, e.g., beads. In some embodiments, the phase transfer media of layer 613a can provide a surface area to volume ratio of at least 500,000 or greater, which provides for a minimum of approximately 1% of the total molecules to be situated at the surface (solvent interface) for immiscible solvents. The large number of surface molecules can advantageously allow for the rapid isolation of compounds with solvents that are miscible, slightly miscible, or completely immiscible.

Exemplary sorbents with affinity for THC include, for example, reverse phase sorbents, such as C18 (Octadecylsilane). Reverse phase sorbents are available commercially including, for example, StrataX™ (Phenomex, Torrance CA), Oasis™ HLB (Waters, Milford, MA) (comprised of hydrophilic N-vinylpyrrolidone and the lipophilic divinylbenzene) and Chromabond HR-X (Macherey-Nagel, Düren, Germany) (a spherical, hydrophobic polystyrene-divinylbenzene resin).

In some embodiments, the materials used for the media of the filter layers, binding layers, and phase transfer layers described herein can be composed of a diatomaceous earth, silica, quartz, glass, alumina, polystyrene, a variety of sands, such as sea sand or loamy sand, or other pulverized materials, such as a powdered metal (metal powder), ceramics, wood of cellulose. In some embodiments, these media can also be functionalized in order to perform chemistry (carbodiimides), buffer a solution (control pH (ex. triethylamine)), or prepare a compound for a subsequent layer in the phase transfer assembly 664 (cyanoborohydride, this can protonate or destroy unwanted things/change a compound to something known). Therefore, the phase transfer assembly 664a, in addition to purification/isolation of compounds, can also be simultaneously used to perform synthetic chemistry. Functionalization of these materials can be performed through well-known chemistry, which varies for each material.

In certain embodiments a selective binding layer, for example, a desiccant material that absorbs aqueous solvent, e.g., water also functions as a phase transfer layer. An exemplary selectively binding layer that also functions as a phase transfer layer is a desiccant material that absorbs aqueous solvent, e.g., water from the liquid sample as the liquid sample passes through the selective binding layer. As organic solvent passes through the selective binding layer, analyte soluble in the organic solvent transfers from the aqueous phase, e.g., aqueous solvent, to the organic phase, e.g., organic solvent. In certain cases, analyte in the liquid sample can bind to the selective binding layer and subsequently be extracted from the selective binding layer as organic solvent passes through the selective binding layer.

### 5. Second Selective Binding Layer

A second selective binding layer, such as a layer of sand 614, placed after the silica can inhibit silica particles from being loaded on the plate.

### 6. Second Filter Layer

A second filter layer, e.g., cotton 615a, positioned after a layer of chromatography medium can contain cannabichromene (CBC), which is used as a control.

### 7. Second Screen Layer

A final screen 616a holds the layers in place in the housing. Any suitable screen can be used, such as a mesh, for example an aluminum screen.

### D. Assembly and Apertures

When assembled, the fluidic chip can contact a face of the chromatography plate comprising the chromatographic medium to form reservoirs on a surface of the plate. In such configurations, chromatography can proceed in a horizontal direction.

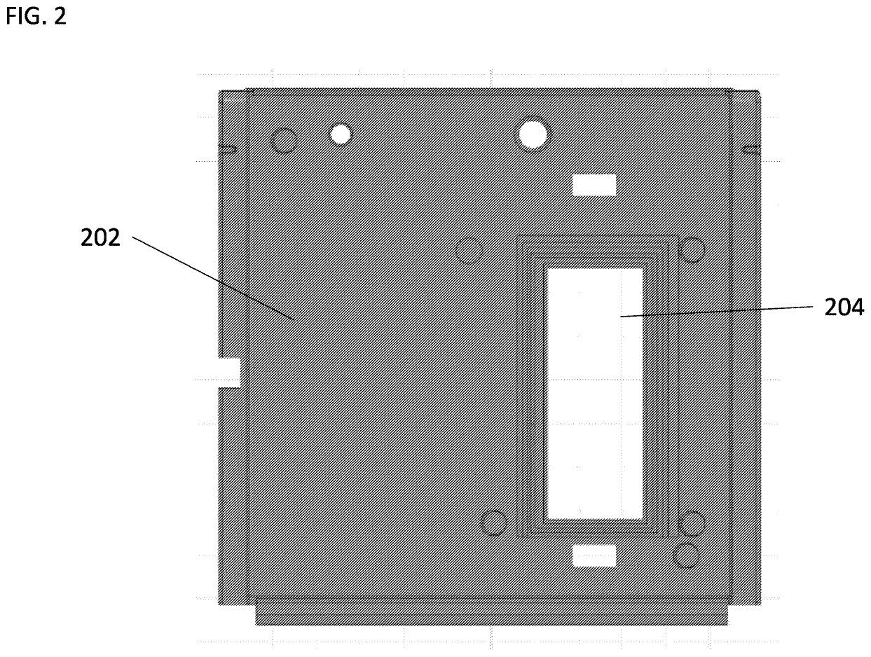

One way to mate the chromatography plate with the fluidic chip is to provide a support, e.g., a tray or holder, that comprises a surface to support the fluidic chip and an indentation in the surface into which the chromatography plate can be inserted such that the fluidic chip rests over the chromatography plate. An exemplary support 202 with a chromatography plate is depicted in FIG. 2.

The one or more apertures in the fluidic chip are positioned to expose a separation area of the chromatography plate such that the separation area can be visualized from above, e.g., with a camera.

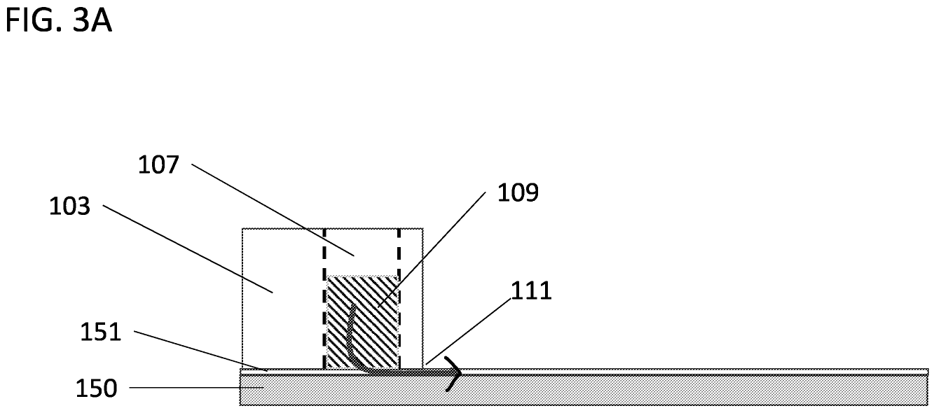

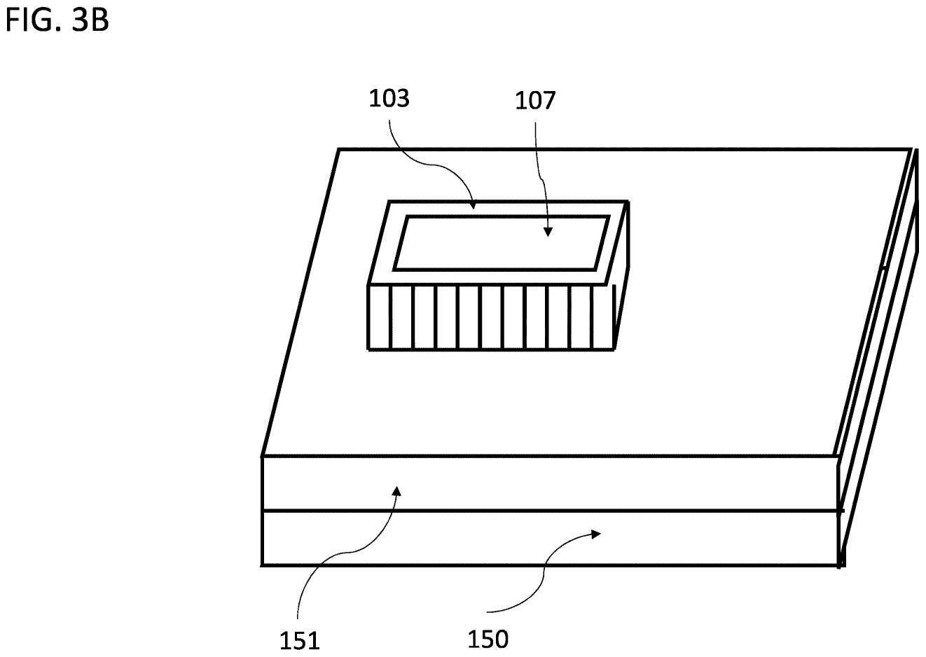

An exemplary area for a large aperture is depicted by the heavy dashed line in FIG. 1. A sectional view of a reservoir is shown in FIG. 3. Fluidic chip 100 with reservoir 107 is mated to chromatography plate comprising substrate 150 and a layer of chromatography medium 151. Liquid, designated by the hatched line is contained in the reservoir, and can only migrate across the plate through the chromatographic medium (movement depicted by arrowed line).

The ports in the fluidic chip through which fluids exit the chip and are deposited onto the plate can be configured as apertures through the plate. One or more apertures can be positioned to expose a surface of the chromatography plate comprising chromatography medium when the plate and the fluidic chip and the chromatography plate are mated. In this configuration the combination of the apertures in the exposed plate surface from a reservoir in which the apertures form a wall of the reservoir and the plate surface forms a floor of the reservoir. As such, fluid deposited in a reservoir cannot flow freely across the surface of the chromatography plate because the fluidic chip pressed against the surface of the plate forms a physical barrier. However, liquid deposited into a reservoir can travel through the chromatography medium and under the barrier and, in this way, migrate across the plate.

### 1. Fluidic Reservoirs

A surface of the fluidic chip can be placed in contact with a surface of the chromatography plate comprising the chromatographic medium. Apertures through the chip expose a surface of the plate, and can function as wells or reservoirs. Liquids deposited in such wells can migrate under the wall of the well that contacts the chromatographic plate through the chromatographic medium.

In certain embodiments, the apparatus further comprises one or more fluidic reservoirs in contact with a surface of the chromatography medium, wherein a liquid phase applied to the fluidic reservoir is in fluid communication with the chromatography medium such that the liquid phase moves via capillary action through the chromatography medium. In certain embodiments, the fluidic reservoirs are not in contact with the edge of the chromatography medium or the edge of the solid substrate and/or wherein the liquid phase does not travel on top of the surface of the chromatography medium.

In certain embodiments, the article comprises (a) a solid substrate, (b) chromatography medium on the surface of the substrate through which liquid phase can travel; (c) one or more fluidic reservoirs in contact with a surface of the chromatography medium, wherein the fluidic reservoir enables application of a liquid phase to the surface of the chromatography medium such that the liquid phase is able to travel through the chromatography medium. In certain embodiments, the fluidic reservoirs are not in contact with the edge of the chromatography medium or the edge of the solid substrate and/or wherein the liquid phase does not travel on top of the surface of the chromatography medium.

The fluidic reservoirs can hold any suitable volume, such as at least any of 100 μL, 200 μL, 300 μL, 400 μL, 500 μL, 600 μL, 700 μL, 800 μL, 900 μL, 1,000 μL, 1,500 μL, or 2,000 μL and/or not more than 5 mL, for example 100 μL to 5 mL. Typically, the fluidic reservoirs are manufactured out of a material resistant and/or compatible with the solvent. Proper materials would be readily identifiable by one of skill in the art.

In certain embodiments, the fluidic reservoirs comprise one or more conduits operably configured to a supply of solvent.

Accordingly, reservoirs can be configured as open wells, such as an open-ended cylinder or block. They can be positioned on the chromatography plate in contact with the chromatography medium. In this way, the chromatography medium will function as a floor of the well, and fluid deposited in the open top of a reservoir will come into contact with the chromatography medium.

The reservoirs can be any suitable shape, such as triangular, rectangular, square, trapezoidal, rhomboidal, pentagonal, hexagonal, heptagonal, octagonal, circular, elliptical, or the like. Typically, the reservoirs comprise an elongated shape such that the solvent generates a linear solvent front along the length of the elongated reservoir.

The reservoirs can be any suitable length, such as at least about any of 2.5 mm, 5 mm, 7.5 mm, 10 mm, 15 mm, 20 mm, 25 mm, 30 mm, 35 mm, 40 mm, 45 mm, 50 mm, 100 mm, 150 mm, 200 mm, or 250 mm and/or no more than about any of 5 mm, 7.5 mm, 10 mm, 15 mm, 20 mm, 25 mm, 30 mm, 35 mm, 40 mm, 45 mm, 50 mm, 100 mm, 150 mm, 200 mm, 250 mm, or 500 mm long, for example between about 2.5 mm and about 500 mm long, preferably, between 50 mm and 500 mm long. The reservoirs can be any suitable width. In certain embodiments the ratio of the length of the reservoir to the width of the reservoir is no more than about any of 2:1, 3:1, 4:1, 5:1, 6:1, 7:1, 8:1, 9:1, 10:1, 15:1, or 20:1.

The reservoirs can be any suitable height, such as at least about any of 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, or 19 mm and/or not more than about any of 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm, for example 1-20 mm.

An exemplary article, e.g., TLC plate, comprising a fluidic reservoir is shown in FIGS. 3A and 3B. The exemplary article comprises a substrate (150) with chromatography medium (151) at least partially coated on the substrate. A fluidic chip (100) forming reservoir (107) is placed on top of the chromatography medium. The fluidic reservoir (107) creates a seal against the chromatography medium (151) such that any fluid placed in the fluidic reservoir (107) doesn't through a junction between the fluidic reservoir (107) and the chromatography medium (151). A side view of the article is shown in FIG. 3A, and an angled view is shown in FIG. 3B. The fluid path (FIG. 3A) of a liquid phase (109) placed into the fluidic reservoir (107) is shown by the arrow, which illustrates the liquid phase traveling radially outward via capillary action from the fluidic reservoir through the chromatography medium (151) but not through the junction (111).

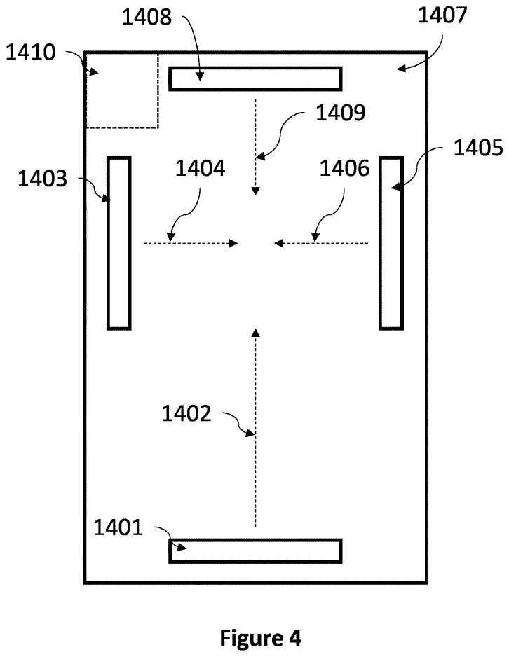

Another exemplary article is shown in FIG. 4. The exemplary article comprises a TLC plate (1407) comprising a substrate at least partially coated in chromatography medium. The TLC plate (1407) comprises at least 2, and optionally 3, fluidic reservoirs (1401, 1403, and 1405), wherein the first fluidic reservoir (1401) is configured to apply solvent in a first dimension (1402), the second fluidic reservoir (1403) is configured to apply solvent in a second dimension (1404), and the optional third fluidic reservoir (1405) is configured to apply solvent in a second dimension (1406) from the opposite direction as the second fluidic reservoir (1403). The second (1403) and third (1405) fluidic reservoirs can be filled with fluid either sequentially or simultaneously. In certain embodiments, the TLC plate (1407) comprises a fourth fluidic reservoir (1408) configured to apply solvent in the first dimension (1409) in a direction opposite of the first fluidic reservoir (1401). In certain embodiments, the first fluidic reservoir also serves as the sample loading zone.

### 2. Pumps

The fluidics assembly comprises one or a plurality of pumps for moving liquids between containers and the cartridge, through application of positive or negative pressure. Any pump known in the art can be used in the instrument of this disclosure. This includes, without exception, syringe pumps, piston pumps peristaltic pumps, and diaphragm pumps.

Syringe pumps and piston pumps move liquids by drawing liquid into a chamber through the movement of a syringe or piston, typically through a first valve, and pushing liquids out of the chamber with a syringe or piston, typically through a second, different valve. These pumps require a motor to move the piston or syringe.

Peristaltic pumps use rollers to compress and release flexible tube the squeezing action creates a positive displacement which propels a liquid through the tube.

Diaphragm pumps include a flexible diaphragm. Movement of the diaphragm by, for example, vacuum, creates negative pressure in the chamber above the diaphragm. Moving the diaphragm for example with positive pressure, creates positive pressure in the chamber above the diaphragm. By positioning valves on either side of the diaphragm liquid can be moved into the chamber by opening a first valve in applying negative pressure to the diaphragm, closing the first valve in opening a second valve, and applying positive pressure to move liquid through the second valve.

### III. Instrument

Also provided herein is an instrument configured to engage the cartridge and automatically perform thin layer chromatography on the cartridge. The instrument can comprise a cartridge interface for engaging the cartridge; a fluidics assembly for moving liquids through the cartridge and onto the chromatography plate; a detector for detecting a signal from the cartridge, e.g., a signal from an analyte or from a calibrant molecule; a fan positioned to direct air to a surface of the TLC plate; a temperature regulator positioned to regulate temperature of a cartridge engaged with the interface; and a computer that comprises operating software to operate the instrument in the performance of thin layer chromatography.

### A. Cartridge Interface

The cartridge interface can comprise a stage for supporting the cartridge and fluid lines configured to engage ports in the cartridge. The fluid lines can be independent. Alternatively, the fluidic lines can be assembled into a fluidics manifold. The fluidics manifold can comprise ports adapted to engage ports in the cartridge.

### 1. Stage

The stage can comprise a platform configured to position and hold the cartridge. The platform can comprise guides for proper positioning of the cartridge.

### 2. Fluidics Manifold

The fluidics manifold can be configured as a block having a plurality of entry points for fluidic lines exiting the pumps, a plurality of through-conduits and a plurality of nozzles that engage the fluidic chip. The fluidics manifold can be movable such that when a cartridge loaded onto the stage, the fluidics manifold moves toward the cartridge to engage the ports, and moves away from the cartridge to disengage the ports.

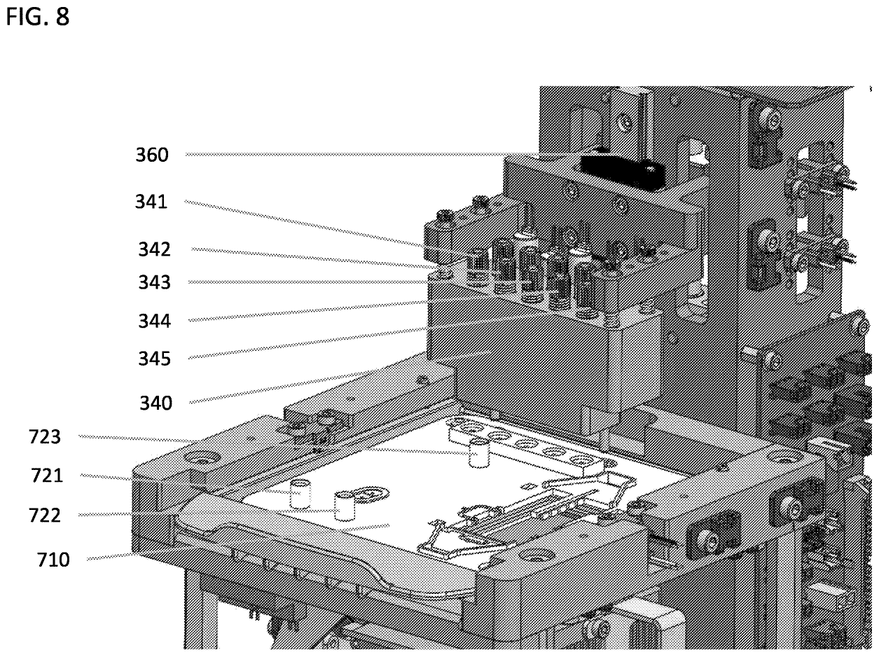

Referring to FIG. 8, exemplary fluidics manifold 340 comprises fluidic connectors 341, 342, 343, 344, and 345, configured to connect with fluid lines from pumps. Stage 710 comprises alignment pegs 721, 722 and 723 that align with holes in the fluidic cartridge to properly positioned the cartridge in the interface. The interface further comprises a mechanical bearing screw-driven lift 360 to move the fluidics manifold along the Y (vertical) axis to engage the fluidic chip.

### B. Fluidics Assembly

The fluidics assembly comprises containers, fluid lines, pumps and, typically, valves (e.g., switchable valves) for moving liquids into, around and out of a cartridge when the cartridge is engaged with the instrument. Typically, positive and/or negative pressure is delivered through a fluidics manifold that is engaged with ports in the engaged cartridge. This includes, for example, application of vacuum to pull sample from a sample cartridge into a fluidic channel of the fluidic chip as well as positive pressure to pump liquids, such as, separating solvent, into the fluidic channel and out port onto the chromatography plate.

### 1. Containers

The fluidics assembly comprises one or a plurality of containers for containing fluids. This includes, for example, glass or plastic containers. The containers or reservoirs can have volumes of between about 10 mL and about 1 L, e.g., between about 50 mL and about 500 mL, or about 100 ml.

### 2. Fluid Lines

Fluid lines, e.g., comprised of plastic tubing, provide a path of fluid communication between containers and ports in the cartridge interface that engage ports in the cartridge.

### 3. Exemplary Fluidics Assembly

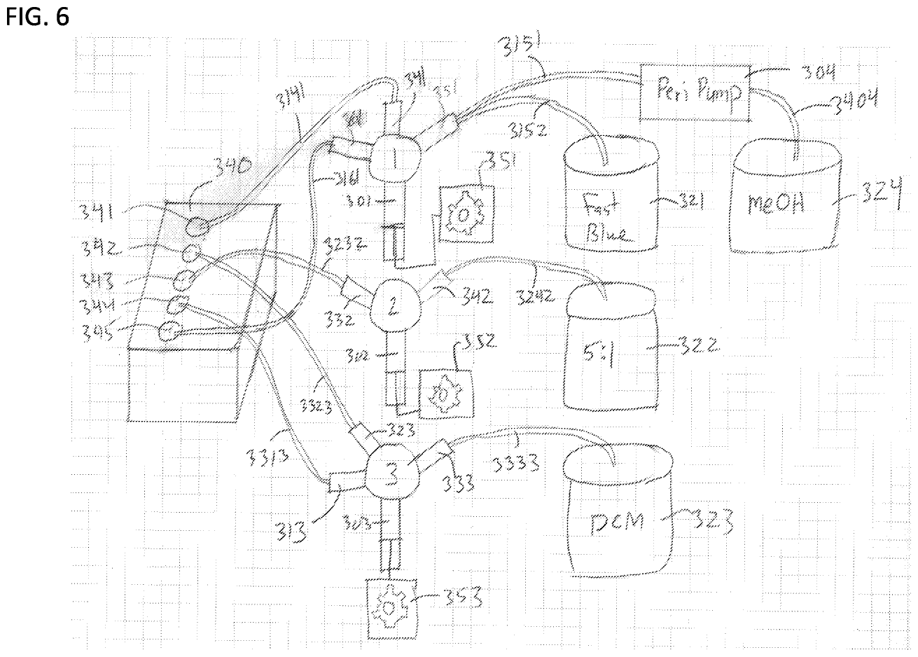

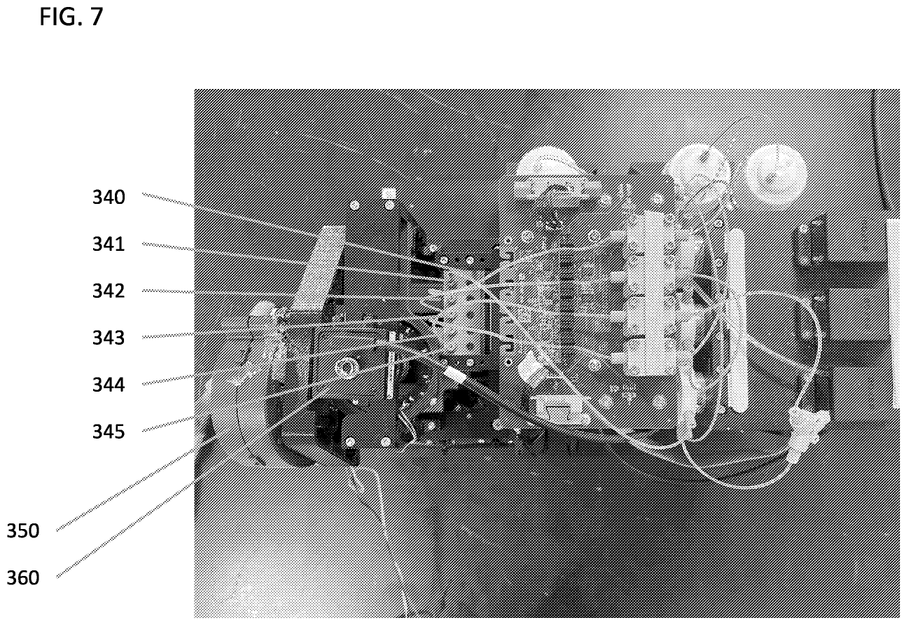

FIGS. 6 and 7 show an exemplary fluidics assembly. The fluidics assembly comprises a plurality of pumps (301, 302, 303, and 304) connected to a plurality of fluid reservoirs (321, 322, 323), and ports (341, 342, 343, 344, and 345) in the fluidics manifold (340), through fluidic conduits, e.g., tubing, and a plurality of switchable valves (361, 362, 363 and 364). Syringe pump 330 is fluidly connected to valve assembly 332. Valve assembly 332 comprises three valves in fluid communication with a DCM reservoir 335, port 340 of the fluidics manifold and port 341 of the fluidic manifold. Pumps fluid from reservoir 332 to fluidic manifold port through with

Syringe pump 303, driven by motor 353, draws fluid from DCM reservoir 323 through fluidic line 3333 and valve 333 of valve assembly 3. Syringe pump 303 can deliver the drawn fluid through valve 323 and fluid line 3323 two port 342 of fluid manifold 340. It also can deliver the drawn fluid through valve 313 and fluid line 3313 two port 344 of fluidic manifold 340.

Syringe pump 302, powered by motor 352, can draw fluid from 5:1 reservoir 322 through fluid line 3242 and valve 342 of valve assembly to. Syringe pump 302 can deliver the drawn fluid through valve 332 and fluid line 3232 two port 343.

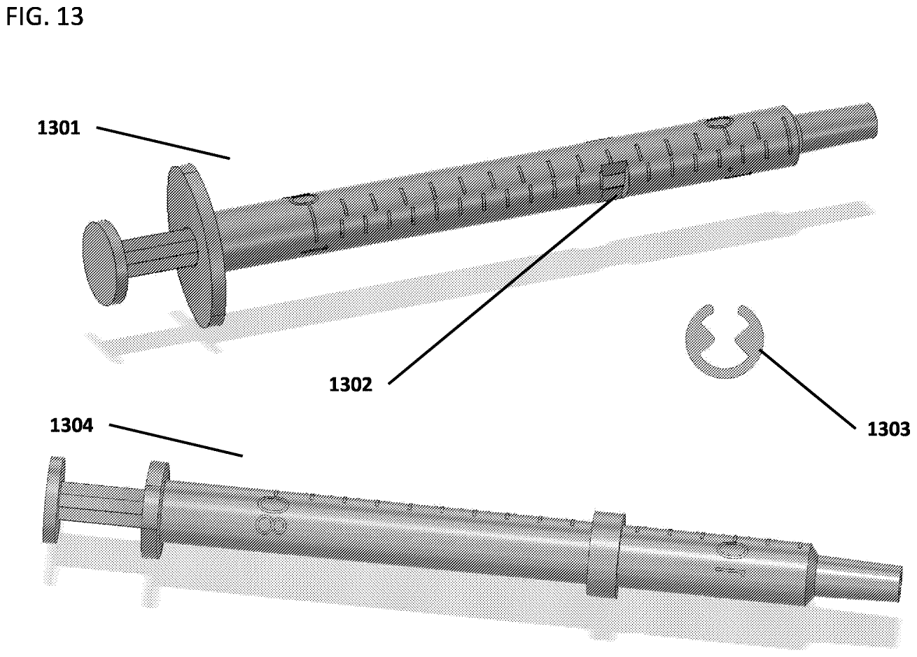

Syringe pump 301, driven by motor 351, can apply vacuum to draw sample fluid into the cartridge through port 101 of the cartridge which is in fluidic communication with port 341 of the fluidics manifold and fluid line 3141 through valve 341 of valve assembly 1. Alternatively, the sample collector can be a syringe affixed to the cartridge via the sample collector port, and the system can comprise an apparatus configured to apply positive pressure to the plunger of the syringe, for example a syringe pump, thereby applying the sample the cartridge. The sample collector syringe can comprise a metering clip affixed to the barrel of the syringe to limit the amount of sample loaded into the syringe by a user. This may improve performance, e.g., reproducibility and reliability of measurements, by providing a means for reproducible loading of sample volume. An exemplary syringe with a metering clip is shown in FIG. 13. In this example, syringe 1301 comprises a notch 1302 configured to accept a metering clip 1303. The syringe assembly 1304 may be used to collect a liquid sample and thereafter engaged with a cartridge for analysis of one or more analytes within the collected liquid sample. Syringe pump 301 also can draw liquid from fast blue reservoir 321 through fluid line 3152 and valve 351. Pump 301 can deliver the drawn fluid through valve 361 and fluid line 3161 two port 345 of the fluidic manifold. In embodiments, where the dye is applied to the cartridge during manufacturing, the fast blue reservoir (321) can be replaced with a solvent reservoir.

Peristaltic pump 304 can pump fluid from methanol reservoir 324 through fluid line 3404 and 3151 through valves 351 and 361 of valve assembly one and fluid line 3161 into port 345 of fluidics manifold 340. Fluid from fast blue reservoir 321 through fluid line 3152 and valve 351 of valve assembly one.

c. Fan

The instrument can further comprise a fan. The fan can be configured to blow air through an aperture in the fluidic chip onto the surface of the chromatographic plate, e.g., towards the stage of the interface when engaged with the cartridge. This can function to evaporate liquids from the chromatographic medium.

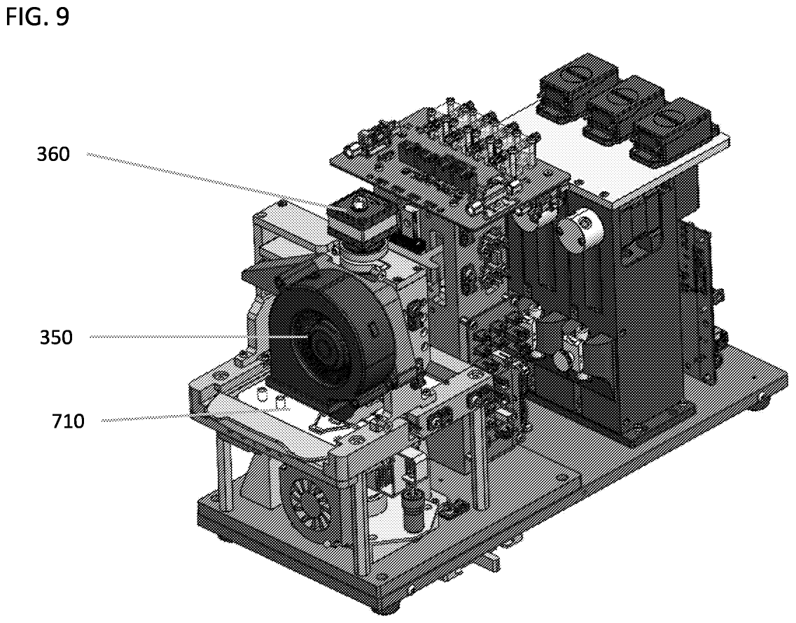

An exemplary fan 350 is depicted in FIG. 7 and FIG. 9.

### D. Temperature Control Assembly

The instrument can comprise a temperature control assembly configured to control the temperature of the chromatography plate. Accordingly, the temperature control element can direct heat or cold towards the stage of the interface when engaged with the cartridge.

The temperature control assembly can be selected from any heating or cooling element suitable for this purpose. These include, without limitation, Peltier devices, resistive heaters, hot air heating, and infrared heating.

Peltier devices, also known as thermoelectric coolers or heaters, use the Peltier effect to generate heating. These devices consist of semiconductor elements that create a temperature difference when an electric current passes through them. By controlling the direction of the current, Peltier devices can generate heat on one side of the plate while absorbing heat on the other side.

Resistive heating involves passing an electric current through a resistive material, such as a wire or a heating element, which generates heat. The heated element then transfers the thermal energy to the plate, raising its temperature.

Hot air heating involves blowing heated air directly onto the plate to raise its temperature. In this method, a heating element, such as an electric coil or a gas burner, warms the air near the plate. The heated air is then directed towards the plate using fans or other means.

Infrared heating involves using infrared radiation to heat the surface of a plate directly. Infrared heaters emit electromagnetic waves in the infrared spectrum, which are absorbed by the plate, converting into heat.

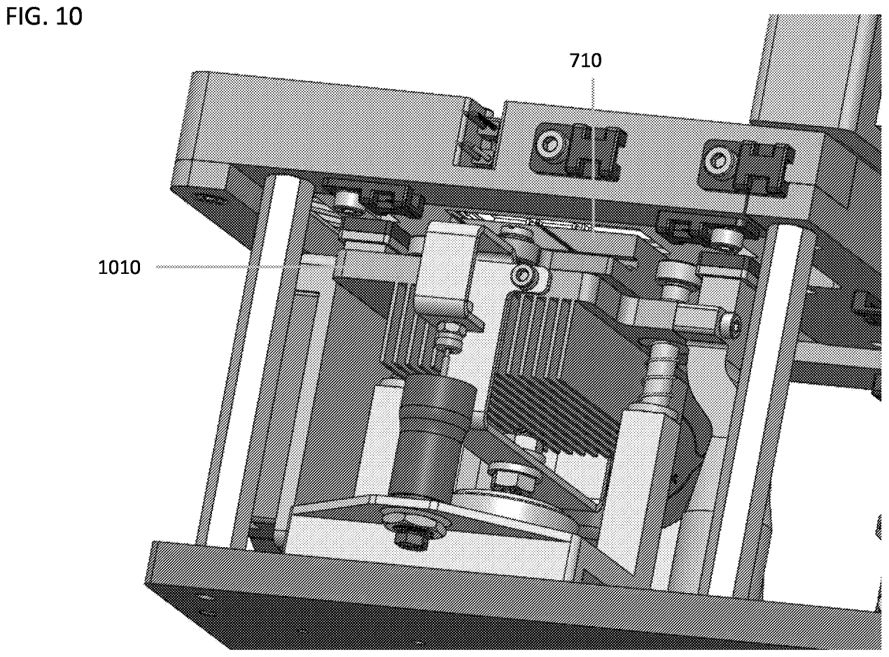

An exemplary Peltier device, 1010 is depicted in FIG. 10.

### E. Detector

The instrument can further comprise a detector to detect a signal from a developed chromatographic plate. The detector can comprise, for example, a camera that detects light emitted by a label.

An exemplary camera 360 is depicted in FIG. 7 and FIG. 9.

### F. Computer

The instrument can further comprise a computer to operate the system. The computer can include one or more of the following elements. A Central Processing Unit (“CPU”) performs calculations and data processing, and executes instructions. Random Access Memory (RAM) is a temporary storage area where the computer stores data and instructions. A Hard Drive or Solid-State Drive (SSD) provides permanent storage for the operating system, software, and data files. Input/Output (I/O) Devices allow interaction with the laboratory instrument and the computer. Examples include keyboards, mice, touchscreens, and various types of sensors or probes that collect data from the instrument. A Display Monitor provides a visual interface for interacting with the computer and viewing data and results from the laboratory instrument. Interface cards can use various protocols such as USB, Ethernet, serial ports, or specialized communication standards to connect with the computer. An operating system (OS) is the software that manages the computer's resources and provides a user-friendly interface. It controls hardware, runs software applications, and facilitates communication between the computer and the laboratory instrument. Instrument-specific Software controls operation of the instrument, data acquisition, analysis, and visualization. The computer also may be connected to a network for data sharing, remote access, or collaboration purposes. Network cards or wireless adapters enable communication with other devices or systems within the laboratory network.

So, for example, the computer can comprise code which, when executed, operates the instrument to perform steps to analyze a sample. This can include, for example, instructing pumps to draw a sample from a sample container, to move a sample through a phase transfer assembly and onto a loading zone of a chromatographic plate by drawing solvent from a solvent container and pushing the solvent into the channel of the fluidic chip, to a separation solvent from a separation solvent container, through the fluidic chip, and into the sample loading zone; moving concentration solvent from a concentration solvent container, through the fluidic chip and into concentration zones on the chromatographic plate.

### G. Power

Electrical elements of the system can operate through the provision of power from a power supply. The power supply can be a battery that is on board the system. Alternatively, the system can have an external power connector, such as a plug or an AC power connector, configured to access an outside power supply, such as an electrical wall socket.

### IV. Methods of Use

Provided herein are automatically performing thin layer chromatography on analytes from a sample.

### A. Samples

The sample can comprise a biological sample, for example a sample sourced from a biological organism. The biological organism can comprise, e.g., a virus, a bacterium, a protist, a eukaryote, an animal, a human, or a plant. The biological sample can comprise saliva, sputum, blood, plasma, serum, urine, stool, cerebral spinal fluid, bile fluid, lymph fluid, or any suitable biological sample tentatively comprising an analyte of interest.

The sample can comprise an environmental sample, for example a sample sourced from the physical environment. The environmental sample can comprise a soil sample, a water sample, an air sample, or a waste sample. In embodiments in which the sample comprises a water sample, the water sample can comprise a reservoir sample, a well water sample, a lake sample, a river sample, an ocean sample, a wetland sample, an ice sample, or any suitable environmental sample tentatively comprising an analyte of interest.

The sample can comprise an industrial sample, for example a sample sourced from an industrial facility or factory, industrial waste, a waste stream from an industrial facility or factory, or an environment directly used for an industrial process. The industrial sample can comprise an industrial product, precursor, or intermediate thereof, for example a production chemical derived from one or more synthetic steps wherein the purity and/or yield of the sample is to be determined, such as dyes, ingredients, preservatives, sweetening agents, food and cosmetic products, or any suitable industrial sample tentatively comprising an analyte of interest.

The sample can comprise an agricultural sample. The agricultural sample is sourced from an agricultural location such as a farm, a field, an animal pen, a body of water for agricultural use, such as a farm site for sea fauna or sea flora or a paddy. The agricultural sample can comprise a soil sample, a plant sample, a livestock sample, a water sample, a food sample, or any suitable agricultural sample tentatively comprising an analyte of interest.

Typically, the sample comprises a liquid sample, for example an aqueous sample comprising an aqueous solvent and tentatively one or more analytes.

### B. Analytes

The analyte of interest can comprise a molecule, for example a molecule sized up to about 50,000 Da, up to about 20,000 Da, up to about 10,000 Da, up to about 5,000 Da, up to about 2,000 Da, or up to about 1,000 Da, for example 50 Da to 50,000 Da. The analyte can be any suitable molecule, for example an organic molecule or an inorganic molecule. The analyte can comprise a small organic molecule, for example a drug with a size of less any of 5,000 Da, 2,000 Da, 1,000 Da, or 500 Da.

The one or more analytes, e.g., compounds of interest, can be, for example, cocaine, an opioid, ayahuasca, a central nervous system depressant, DMT, GHB, a hallucinogen, heroin ketamine, KHAT, LSD, MDMA (ecstasy/molly), mescaline (peyote), methamphetamine, an over-the-counter medicine, including but not limited to dextromethorphan, loperamide, and the like, PCP, a prescription medication, a stimulant, psilocybin, rohypnol (flunitrazepam), saliva, steroids (anabolic), a cannabinoid or a variant thereof, cathinone (bath salts), or any other suitable analyte of interest.

The analyte can be one or more controlled substances. The analyte can be a drug. The analyte can be a cannabinoid. The cannabinoid can comprise 10-ethoxy-9-hydroxy-delta-6a-tetrahydrocannabinol, 10-oxo-delta-6a-tetrahydrocannabinol (OTCH), 2-arachidonoylglycerol (2AG), 2-arachidonyl glyceryl ether, 8,9-dihydroxy-delta-6a-tetrahydrocannabinol, anandamide (AEA), cannabichromanon (CBCN), cannabichromene (CBC), cannabichromenevarin (CBCV), cannabichromenic Acid (CBCA), cannabichromevarinic acid (CBCVA), cannabicitran (CBT-C), cannabicyclol (CBL), cannabicyclolic acid (CBLA), cannabicyclovarin (CBLV), cannabidiol (CBD), cannabidiol monomethylether (CBDM), cannabidiolic acid (CBDA), cannabidiorcol (CBDC1), cannabidiorcol (CBN-C1), cannabidivarin (CBDV), cannabidivarinic acid (CBDVA), cannabielsoic acid A (CBEA-A), cannabielsoic acid B (CBEA-B), cannabielsoin (CBE), cannabifuran (CBF), cannabigerol (CBG), cannabigerol monomethylether (CBGM), cannabigerolic Acid (CBGA), cannabigerolic acid monomethylether (CBGAM), cannabigerovarin (CBGV), cannabigerovarinic acid (CBGVA), cannabiglendol-C3, cannabinodiol (CBND), cannabinodivarin (CBV), cannabinodivarin (CBVD), cannabinol (CBN), cannabinol methylether (CBNM), cannabinol-C2 (CBN-C2), cannabinol-C4 (CBN-C4), cannabinolic acid (CBNA), cannabiripsol (CBR), cannabitriol (CBT), cannabitriolvarin (CBTV), dehydrocannabifuran (CBFD), delta-8-tetrahydrocannabinol (Δ8-THC), delta-8-tetrahydrocannabinolic acid (Δ8-THCA), delta-9-cis-tetrahydrocannabinol (CIS-THC), delta-9-tetrahydrocannabinol (Δ9-THC), delta-9-tetrahydrocannabinol-C4 (Δ9-THC-C4), delta-9-tetrahydrocannabinolic acid A (Δ9-THCA-A), delta-9-tetrahydrocannabinolic acid B (Δ9-THCA-B), delta-9-tetrahydrocannabinolic acid C4 (Δ9-THCA-C4), delta-9-tetrahydrocannabiorcol (Δ9-THCA-C1), delta-9-tetrahydrocannabiorolic acid C1 (Δ9-THCA-C1), delta-9-tetrahydrocannabivarin (THCV), delta-9-tetrahydrocannabivarinic acid (THCVA), lysophosphatidylinositol (LPI), N-arachidonoyl dopamine (NADA), tetrahydrocannabinol (THC), trihydroxy-delta-9-tetrahydrocannabinol (TRIOH-THC), virodhamine (OAE), or a variant thereof. The variant can comprise either a natural or synthetic variant.

### C. Methods

Provided herein are methods for isolating an analyte from a sample matrix and measuring the analyte by thin layer chromatography. A “sample matrix” refers to a sample comprising many components, impurities and interfering compounds in addition to the analyte. Exemplary sample matrices are biological samples (e.g., blood, plasma, serum, urine, saliva, tissue extracts, or cell culture media), environmental samples (e.g., water (including drinking water, surface water, wastewater), soil, sediment, air particulate matter, or biota), industrial samples (e.g., samples from chemical manufacturing, oil and gas industry, polymers, cosmetics, or consumer products), and food samples (fruits, vegetables, meat, dairy products, grains, or processed foods).

A sample matrix of a biological sample can comprise, for example, particulate matter, undigested food, proteins, cells, and mucins. The methods of this disclosure can separate an analyte, such as a small organic molecule, from these matrix components.

Methods of isolating an analyte from a sample matrix and measuring the analyte by thin layer chromatography include, (1) separating the analyte from first interfering substances by automatically passing the sample through a phase transfer assembly that captures or retards first interfering substances; (2) separating the analyte and remaining interfering substances by thin layer chromatography by automatically depositing the light remaining interfering substances onto a thin layer chromatography plate and using a separation medium to develop the plate; and (3) automatically detecting the separated analyte.

Separating the analyte from first interfering substances can be performed by using a pump to move a sample matrix into a phase transfer assembly comprising a fluidic channel comprising a phase transfer media, optionally by moving the sample matrix into a first fluidic channel by vacuum and then using a liquid under pressure to move the sample into the phase transfer assembly.

Separating the analyte and remaining interfering substances by thin layer chromatography can be performed by eluting the analyte and remaining interfering substances from the phase transfer assembly by first, using a pump to move a liquid phase over the phase transfer assembly to elute the analyte, and depositing the analyte and remaining interfering substances onto a loading zone of a thin layer chromatography plate; and, second, using a pump to move a separation solvent onto the loading zone. This will result in migration of the separation solvent through the chromatography medium and across the thin layer chromatography plate, separating the analyte from other interfering substances by chromatography.

In an optional step, the analyte may move across the chromatography medium as a band. In this case, the band of analyte can be compressed or concentrated by the using a pump to deposit concentration solvent to the chromatography plate lateral to the band. The concentration solvent will migrate toward the center of the band, moving analyte molecules with it.

Automatically measuring the analyte can comprise using a computer-controlled camera to take an image of the chromatography plate and using software to determine the amount of analyte based on the intensity of the image of the analyte.

These steps can both be integrated and automated. They can be integrated in that all the steps can be performed using a single cartridge in a single system. They are automated in that after a sample is provided to the cartridge, the system performs the remaining steps to detection without outside intervention, e.g., without human intervention. The deposition of fluids can occur without the use of pipettes to move liquids from one location to another. That is, liquid can move from a container to the chromatography plate in a continuous fluidic line. They can be automated by the use of mechanical and electrical devices, e.g., motorized pumps, heaters, fans and cameras, controlled by a software product on the computer. That is, once initiated, the steps can proceed to the end without human intervention.

An exemplary method involves the following steps:

A sample is collected in a sample container that comprises an outlet adapted to engage a sample port of a cartridge. The sample can be, for example, a saliva sample collected by spitting into the sample container. The sample contains an analyte to be detected, for example, a controlled substance, such as THC. The sample container is then engaged with the sample port in the cartridge through any suitable connection, for example a luer fitting.

The cartridge, containing the sample container, is then engaged with the cartridge interface of the system. The fluidics manifold is then engaged with the various ports in the fluidic cartridge.

The system uses vacuum generated by the fluidics assembly to pull sample, e.g. saliva, from the sample container into a fluidic channel of the fluidic cartridge.

An organic solvent, such as dichloromethane, is drawn by a pump from a liquid reservoir and pumped into the fluidic cartridge. The fluidic channel leading from the first solvent reservoir intersects the channel comprising the sample. Thereby, a specific volume of sample, represented by the volume from the intersection to the intersection with the channel leading from the sample entrance, is pushed into the phase transfer assembly.

A phase transfer assembly filters out particulate matter, captures water from the sample and binds analyte to the phase transfer media. Continued pumping of the organic solvent through the phase transfer assembly to elute the analyte from the phase transfer media and deposits it into the sample loading reservoir of the chromatography plate.

Separating solvent is then deposited into the sample reservoir. This is done by drawing separating solvent from a fluid reservoir using a pump of the fluidic assembly and moving the liquid into the solvent port of the fluidic chip. The separating solvent is then moved through a fluidic channel in the fluidic chip and into the sample reservoir.

The separating solvent spreads across the sample reservoir and migrates as a front through the chromatography medium by capillary action, separating molecules, including the analyte, from each other.

After separation, the band of analyte is compressed by application of a concentrating solvent on either side of the band. This is done by drawing liquid from a solvent reservoir using a pump of the fluidics assembly and moving the draw liquid into one or more concentration ports in the fluidic chip, through fluidic channels in the chip, and depositing the concentration solvent in concentration wells on the chromatography plate. Concentration solvents move toward each other compressing a concentrating the analyte into a narrower band or spot.

A suitable detector, e.g., a camera, can be used to monitor how far the solvent, e.g., separating and/or concentrating solvent, has traveled. This can be performed by taking an image of the TLC plate, identifying a solvent front, and measuring the distance between the starting position of the sample and the position of the solvent front. Additionally or alternatively, multiple images can be taken a velocity of the solvent front can be calculated using both distance and time between image collections. The distance and/or velocity measurements of the solvent front can be used as part of a control system to optimize separation and/concentration steps. This can help improve reproducibility and performance of the system.

An exemplary method includes using a camera and control system to determine if additional solvent needed. In this case, images can be taken during analysis to determine how far the solvent has traveled. Additional solvent is added until the solvent front has reached a predetermined location, for example to optimize separation and/or concentration steps. Reproducible positioning can help to prevent variation in the manufacturing of TLC plates from having an impact on system performance. Measuring solvent front position and/or velocity can improve assay-to-assay and/or system-to-system reproducibility. In certain embodiments, the use of systems and methods to measure solvent front position and/or velocity reduces the coefficient of variation of assay-to-assay and/or system-to-system measurements of the amount of one or more analytes to less than any of 40, 30, 20, 10, 9, 8, 7, 6, 5, 4, 3, 2, or 1%.

Another exemplary method includes using a camera and a control system to measure the velocity of the solvent front. In this case, the system can take pictures of the TLC plate at defined intervals, e.g., every 5 seconds. The system will calculate how quickly the solvent is moving on the TLC plate and, optionally adjust the rate of solvent addition to control the velocity of the solvent front. Control of the velocity of the solvent front can improve performance in both separation and concentration steps as well as improve reproducibility of assays. This can further help improve performance in light of variations in the TLC plates, temperature, altitude, and the like.

Additionally or alternatively, a reference chemical may be added to the sample as an internal standard. An exemplary method uses a camera and a control system to measure the position of the internal standard and thus monitor progression of the separation and/or concentration steps. In certain embodiments, the internal standard is added to the phase transfer assembly during manufacturing. An exemplary cartridge can include a suitable amount, e.g., about any of 5, 10, 25, 50, 100, 150, 200, 300, 400, or 500 ug, of a reference chemical within the phase transfer assembly, for example about 150 ug of a reference chemical added to the cotton of the phase transfer assembly of the cartridge. Additionally, the internal standard can provide an indication that the methods completed correctly, for example an improper final location on the TLC plate of the internal standard could indicate a failure of one or more steps in the method suggesting that the sample need to be rerun.

The analyte is stained for detection using a dye. For example, Fast Blue B binds cannabinoids. It also absorbs light in the range of 500 nm to 600 nm, with an absorption peak of about 550 nm. Consequently, any suitable amount of the dye can be deposited on the chromatography plate and allowed to bind the analytes. Then, shining an LED that emits light in the 500 nm to 600 nm range causes the dye to become visible. Alternative dyes include fast blue bb salt, fast black k, fast red TR salt, and fast garnet GBC sulfate salt. Solvents for the dye include, for example, methanol, hexanes, water, acetonitrile, dichloromethane, dibromomethane, chloroform, toluene, acetone, methyl-thf, pentane, cyclopentyl methyl-ether, ethyl acetate, and 1-octanol.

The analyte spot is then detected using the detector, such as a camera. The camera takes an image of the spot as well as calibrants. The image is uploaded to a computer. The computer uses software to determine the intensity of the signal from the analyte which is a function of the amount of analyte on the chromatography plate. Then, based on the intensity of signal from the calibrants, software generates a measure of the amount of analyte on the plate. Spots of known concentration of the analyte are added to the plate as calibrants before the test is performed. For example, spots of 10 ng, 50 ng and 100 ng are added to the plate. When the dye is added to the plate each know spot will have a different intensity, The intensities of the spots and correlated concentration are fitted to a straight line. The unknow is compared to the line and the intensity is determined from this analysis.

### Exemplary Embodiments

1. A method comprising:
(a) performing thin layer chromatography (“TLC”) on one or more analyte molecules by:
   (i) moving a liquid sample comprising the analyte molecules into a fluidic channel of a fluidic chip;
   (ii) moving the liquid sample through a phase transfer assembly comprised in a fluidic channel internal or external to the fluidic chip to separate analyte molecules from contaminants;
   (iii) delivering the separated analyte molecules from a port in the fluidic chip to a sample loading zone of a TLC plate comprising chromatography medium on a solid substrate;
   (iv) moving a separating solvent through a fluidic channel in the fluidic chip and delivering the separating solvent from the fluidic chip to the sample loading zone; and
   (v) developing the TLC plate by allowing the separating solvent to migrate with the analyte molecules into a separation zone that extends in the first dimension from the sample loading zone;
(b) optionally, concentrating the analyte by:
   (i) moving a concentrating solvent through one or more fluidic channels in the fluidic chip and delivering the concentrating solvent from the fluidic chip to one or more concentrating solvent loading zones on the TLC plate, wherein the one or more concentrating solvent loading zones are positioned in the separation zone and lateral relative to the analyte molecules; and
   (ii) further developing the TLC plate by allowing the concentrating solvent to migrate through the chromatography medium in a dimension orthogonal or oblique to the first dimension;
(c) optionally, tagging the analyte molecules with a detectable tag by:
   (i) moving a fluid comprising a detectable tag through one or more fluidic channels in the fluidic chip and delivering the fluid comprising the tag to separation zone, and along the tag detect analyte molecules; and
(d) detecting, with a detector, a signal from the TLC plate indicating the presence of the analyte molecules.

2. The method of embodiment 1, wherein:
(i) the fluidic chip comprises a sample inlet port, a negative pressure port, a positive pressure port, and a liquid delivery port, fluidically connected to each other through a network of fluidic channels, wherein, a channel between the sample inlet port and the positive pressure port is intersected by a channel from the negative pressure port and a channel from the liquid delivery port, thereby defining a channel segment;
(ii) using negative pressure applied to the negative pressure port, to draw a volume of the liquid sample, sufficient to fill the channel segment; and
(iii) using positive pressure applied to the positive pressure port to push the volume through the phase transfer assembly and extract analyte from the volume.

3. The method of embodiment 1, wherein moving the liquid sample into the fluidic channel comprises engaging a sample container containing the sample liquid with a port in the fluidic chip.

4. The method of embodiment 1, wherein moving the liquid sample into the fluidic channel comprises drawing the sample through a sample port in the fluidic chip by applying negative pressure to a pressure port in the fluidic chip in communication with the sample port.

5. The method of embodiment 1, wherein moving liquids through fluidic channels comprises pumping the liquids with one or more pumps that apply positive pressure to the channels.

6. The method of embodiment 1, wherein delivering the analyte molecules to the sample loading zone comprises moving a polar, organic solvent through the fluidic channels, e.g., in an amount between about 0.1 mL and about 1 mL.

7. The method of embodiment 1, wherein developing the TLC plate comprises using a fan to evaporate solvent from a surface of the TLC plate.

8. The method of embodiment 1, wherein developing the TLC plate comprises using a temperature regulation device to heat the TLC plate.

9. The method of embodiment 1, wherein the sample comprises an environmental sample.

10. The method of embodiment 1, wherein the sample comprises a biological sample, e.g., saliva.

11. The method of embodiment 1, wherein the analyte comprises a psychoactive drug, e.g., THC.

12. The method of embodiment 1, wherein the analyte is present in the mixture at a concentration of no more than 1%.

13. The method of embodiment 1, wherein the analyte comprises THC and the detectable tag comprises fast blue.

14. The method of embodiment 1, wherein detecting comprises using a camera to detect an image from the TLC plate.

15. The method of embodiment 1, wherein detecting comprises quantifying an amount of detect analyte.

16. The method of embodiment 1, wherein detecting comprises detecting an image from the TLC plate they camera, digitizing the image and using computer software to quantify an amount of the analyte in the digitized image.

17. The method of embodiment 1, comprising depositing on the thin-layer chromatography plate as little as any of 50 ng, 20 ng, 5 ng, 2 ng, 1 ng or 0.2 ng of an analyte (e.g., THC) and detecting the analyte.

18. The method of embodiment 1, comprising passing between 200 microliters and 1000 microliters of a sample comprising an analyte in a concentration as low as any of 125 nanograms per milliliter, 50 nanograms per milliliter, 12.5 nanograms per milliliter, 2.5 nanograms per milliliter or 0.5 nanograms per milliliter, through the phase transfer assembly, and detecting the analyte.

19. The method of embodiment 1, performed in no more than any of 1 hour, 45 minutes, 30 minutes, 15 minutes or 5 minutes.

20. The method of embodiment 1, performed by a system of embodiment 48.

21. A cartridge for performing thin layer chromatography, comprising a fluidic chip mated to a thin layer chromatography (“TLC”) plate; wherein:
(a) the TLC plate comprises a surface comprising chromatographic medium, the surface comprising a sample loading zone, a separation zone extending in a first dimension from the sample loading zone, and, optionally, one or more concentrating solvent loading zones positioned in the separation zone; and
(b) the fluidic chip comprises:
   (i) a first fluidic circuit comprising ports connected by one or more internal and, optionally, external, fluidic channels, including a sample inlet port, at least one pressure port, and a liquid delivery port; wherein the first fluidic circuit is configured to receive, by negative pressure delivered through a pressure port, a sample from the sample inlet port into a fluidic channel, and to deliver, by positive pressure delivered through a pressure port, at least part of the sample through a fluidic channel comprising a phase transfer medium, and out the liquid delivery port onto the sample loading zone; and
   (ii) a second fluidic circuit comprising ports connected by one or more fluidic channels, including a separating solvent intake port and a separating solvent delivery port, wherein the separating solvent delivery port is positioned to deliver separating solvent onto the sample loading zone, wherein the second fluidic circuit optionally shares ports and/or fluidic channels with the first fluidic circuit;
   (iii) optionally, one or more third fluidic circuits comprising ports connected by fluidic channels, including one or more concentrating solvent intake ports and one or more concentrating solvent delivery ports, wherein the one or more concentrating solvent delivery ports are positioned to deliver concentrating solvent to the one or more concentrating solvent loading zones;
   (iv) optionally, one or more fourth fluidic circuits comprising ports connected by one or more fluidic channels, including a dye intake port and a dye delivery port, wherein the dye delivery port is positioned it to delivery dye onto the separation zone; and
   (v) an aperture through the fluidic chip which exposes the separation zone.

22. The cartridge of embodiment 21, wherein the one or more concentrating solvent loading zones are positioned lateral relative to the sample loading zone.

23. The cartridge of embodiment 21, wherein the TLC plate comprises calibrants spotted on the chromatography medium.

24. The cartridge of embodiment 21, wherein the TLC plate comprises a solid substrate selected from glass, glass, quartz, metal, aluminum, and plastic.

25. The cartridge of embodiment 21, wherein the chromatography medium is selected from silica, alumina, cellulose, and polyamide.

26. The cartridge of embodiment 21, wherein the first fluidic circuit comprises a branched fluidic channel connected to the sample inlet port, wherein a first branch connects with the first pressure port through a branched channel, and a second branch connects with the liquid delivery port.

27. The cartridge of embodiment 21, wherein the fluidic channel comprising the phase transfer medium is external to the fluidic chip and connected to internal channels through ports.

28. The cartridge of embodiment 21, wherein the fluidic channel comprising the phase transfer medium is internal to the fluidic chip.

29. The cartridge of embodiment 21, wherein the phase transfer assembly comprises a filter for filtering particulate matter, a hydrophilic resin and a solid phase extraction.

30. The cartridge of embodiment 29, wherein the solid phase extraction medium comprises a reverse phase resin.

31. The cartridge of embodiment 21, wherein the second fluidic circuit is not in fluid communication with the first fluidic circuit.

32. The cartridge of embodiment 21, wherein the fluidic chip comprises the one or more third fluidic circuits.

33. The cartridge of embodiment 32, wherein the one or more third fluidic circuits comprise first and second concentrating solvent delivery ports.

34. The cartridge of embodiment 33, wherein the fluidic chip comprises a single third fluidic circuit.

35. The cartridge of embodiment 21, wherein the fluidic chip comprises the one or more fourth fluidic circuits.

36. The cartridge of embodiment 35, wherein the fluidic chip comprises a single fourth fluidic circuit.

37. The cartridge of embodiment 21, comprising one or more loading reservoirs in contact with the surface over the sample loading zone, wherein the liquid delivery port and the solvent delivery port are in fluidic communication with the one or more loading reservoirs.

38. The cartridge of embodiment 37, wherein the liquid delivery port and the solvent delivery port communicate with different loading reservoirs.

39. The cartridge of embodiment 21, comprising one or more concentrating reservoirs in contact with the surface over the one or more concentrating solvent loading zones, wherein the one or more concentrating solvent delivery ports are in fluidic communication with the one or more loading reservoirs, optionally wherein the concentrating reservoirs have an elongate shape oriented substantially parallel to the first dimension.

40. The cartridge of embodiment 39, wherein the concentrating reservoirs have an aspect ratio in an elongate dimension of at least 5:1 or 10:1.

41. The cartridge of embodiments 37 or 39, wherein one or more of the reservoirs are formed at least in part in the fluidic chip, wherein contact between the fluidic chip and the TLC plate surface forms a barrier whereby liquid in a reservoir migrates under the reservoir and through the chromatography medium.

42. The cartridge of embodiment 41, wherein the reservoir comprises an elongate, e.g., linear aspect that allows separation medium to travel as a substantially linear front.

43. The cartridge of embodiment 21, comprising a holder configured to support the fluidic chip and the TLC plate.

44. The cartridge of embodiment 43, wherein the holder comprises a portion configured to support the TLC chip that is recessed with respect to a portion configured to support the fluidic chip.

45. The cartridge of embodiment 21, further comprising a sample collector engaged with the sample inlet port.

46. The cartridge of embodiment 21, wherein the fluidic chip comprises mated first and second pieces, wherein one or both of the pieces comprise open channels on a face of the piece and/or apertures through the piece, wherein needing the first and second pieces closes the channels and puts to channels in communication with a plurality of the apertures.

47. The cartridge of embodiment 46, wherein at least one aperture through a piece communicates with an open channel on a face of the piece, or, at least one aperture through a piece communicates with an open channel on the other piece when the pieces are mated.

48. A system comprising:
(a) a cartridge interface for engaging a cartridge, wherein the cartridge interface comprises:
   (i) a stage for holding the cartridge; and
   (ii) a fluidics manifold comprising a plurality of fluidic ports adapted to engage ports in a cartridge when held by the holder;
(b) a fluidics assembly comprising:
   (i) a plurality of containers for containing fluids;
   (ii) a plurality of fluid lines; and
   (iii) one or more pumps configured to pump liquids from the containers, through the fluid lines, to the fluidic ports, and to apply positive or negative pressure through at least one fluid line to at least one fluidic port;
   (iv) optionally, one or more valves configured to switch fluidic connections between the containers and fluid lines leading to the fluidics manifold;
(c) a detector configured to detect a signal from the cartridge;
(d) optionally, a fan positioned to direct air to a surface of a thin layer chromatography plate of a cartridge when the cartridge is engaged with the cartridge interface.
(e) optionally, a temperature regulator positioned to regulate temperature of a cartridge engaged with the interface;
(f) a computer comprising operating software to control actions of the cartridge interface, the fluidics assembly and the detector, and, when present, the fan and the temperature regulator; and
(g) optionally, a cartridge engaged with the cartridge interface.

49. The system of embodiment 48, wherein the stage is slidably attached to a chassis of the system to allow placement of the cartridge on the stage when the stage is extended from the interface, and to allow engagement of the fluidics manifold when the stages retracted into the system.

50. The system of embodiment 48, wherein the fluidics manifold comprises at least any of three, four, five, six, seven, or eight fluidic ports.

51. The system of embodiment 48, wherein the plurality of containers include containers differently containing one, two, three, four, or five liquids selected from a separating solvent, an organic polar solvent, a concentrating solvent, and a dye.

52. The system of embodiment 51, wherein the separating solvent is selected from dichloromethane, methanol, hexanes, water, acetonitrile, dibromomethane, chloroform, toluene, acetone, methyl-thf, pentane, cyclopentyl methyl-ether, ethyl acetate, and 1-octanol.

53. The system of embodiment 51, wherein the organic polar solvent is selected from dichloromethane, methanol, hexanes, water, acetonitrile, dibromomethane, chloroform, toluene, acetone, methyl-thf, pentane, cyclopentyl methyl-ether, ethyl acetate, and 1-octanol.

54. The system of embodiment 51, wherein the concentrating solvent is selected from dichloromethane, methanol, hexanes, water, acetonitrile, dibromomethane, chloroform, toluene, acetone, methyl-thf, pentane, cyclopentyl methyl-ether, ethyl acetate, and 1-octanol.

55. The system of embodiment 51, wherein the dye is selected from fast blue, fast blue bb salt, fast black k, fast red TR salt, and fast garnet GBC sulfate salt.

56. The system of embodiment 48, wherein one or more of the pumps is a syringe pump.

57. The system of embodiment 48, wherein one of the pumps is a peristaltic pump.

58. The system of embodiment 48, wherein the one or more pumps is at least any of two, three, or four pumps.

59. The system of embodiment 48, wherein the fluidics assembly comprises the one or more valves.

60. The system of embodiment 48, wherein the detector comprises a camera.

61. The system of embodiment 48, wherein the detector comprises a source of light in the range of 500 nm to 600 nm.

62. The system of embodiment 48, comprising the fan.

63. The system of embodiment 48, comprising the temperature regulator.

64. The system of embodiment 63, wherein the temperature regulator comprises a Peltier.

65. The system of embodiment 48, wherein a cartridge is engaged with the cartridge interface.

66. The system of embodiment 65, wherein the cartridge comprises a cartridge of embodiment 21.

67. The system of embodiment 65, wherein the cartridge comprises a fluidic chip mated with a thin layer chromatography plate, wherein the chip comprises an aperture exposing a chromatography surface of the TLC plate and wherein the system comprises the detector which is configured to detect a signal from the surface.

68. A method comprising performing thin layer chromatography (“TLC”) by moving a sample from a fluidic channel in a fluidic chip onto a sample loading zone of a thin layer chromatography plate; moving a separation solvent from a fluidic channel in a fluidic chip onto the sample loading zone; and developing the TLC plate by allowing the separating solvent to separate analytes in a separation zone of the TLC plate and a first dimension.

69. The method of embodiment 68, further comprising, moving concentration solvent from one or more fluid channels in the fluidic chip onto one or more concentration solvent loading zones positioned in the separation zone lateral relative to the sample loading zone; and further developing the TLC plate by allowing the concentrating solvent to concentrate analytes in the separation zone in a second dimension.

70. The method of embodiment 68 or 69, further comprising, moving detection dye from the channel in the fluidic chip onto the separation zone and allowing the dye to tag the analytes.

71. A system comprising:
(a) a computer comprising:
   (i) a processor; and
   (II) a memory, coupled to the processor, the memory comprising a module comprising:
(1) computer executable instructions for implementing the method of embodiment 20.

72. A cartridge comprising:
(1) a fluidic chip comprising:
   (a) a first fluidic channel in fluidic communication with a first inlet and a first outlet;
   (b) a second fluidic channel in fluidic communication with a second inlet and a second outlet; and, optionally,
   (c) a third fluidic channel in fluidic communication with a third inlet and a third outlet, wherein the first, second and third fluidic channels are disposed in a substantially planar substrate disposed between a first layer and a second layer; and
(2) a chromatography plate attached to at least a portion of the fluidic chip, wherein the first, second, and, optionally, third outlets are in fluid communication with the surface of the chromatography plate.

73. A cartridge comprising:
(1) a fluidic chip comprising:
   (a) a plurality of inlets,
   (b) a plurality of outlets, and
   (c) a plurality of channels in the substrate in fluid communication with at least one inlet and at least one outlet; and
(2) a thin layer chromatography plate in contact with at least a portion of the fluidic chip such at least two of the plurality of outlets are in fluid communication with the plate.

74. A cartridge comprising:
(1) a fluidic chip comprising:
   (a) a plurality of reservoirs, each reservoir configured receive a liquid phase, and
   (b) a plurality of channels in fluid communication with the plurality of reservoirs; and
(2) a thin layer chromatography plate in contact with at least a portion of the fluidic chip such that the plurality of reservoirs are in fluid communication with the plate.

75. A cartridge comprising:
(1) a conduit comprising at least one chromatography resin;
(2) a thin layer chromatography plate; and
(3) a fluidic chip comprising at least one channel, wherein the chromatography resin and the plate are in fluid communication through the channel.

76. A system comprising:
(A) a manifold for inserting a cartridge;
(B) a cartridge inserted in the manifold, the cartridge comprising:
   (1) a fluidic chip comprising:
      (a) a first fluidic channel in fluidic communication with a first inlet and a first outlet;
      (b) a second fluidic channel in fluidic communication with a second inlet and a second outlet; and, optionally,
      (c) a third fluidic channel in fluidic communication with a third inlet and a third outlet, wherein the first, second and third fluidic channels are disposed in a substantially planar substrate disposed between a first layer and a second layer; and
   (2) a chromatography plate attached to at least a portion of the substrate, wherein the first, second, and, optionally, third outlets are in fluid communication with the surface of the chromatography plate; and
(C) a first conduit in fluidic communication with the first inlet and a first valve, a second conduit in fluidic communication with the second inlet and a second valve, and, optionally, the third inlet.

77. A method comprising:
(A) delivering a sample comprising at least one analyte to a first inlet of a fluidic chip, wherein the fluidic chip comprises:
   (a) a first fluidic channel in fluidic communication with the first inlet and a first outlet;
   (b) a second fluidic channel in fluidic communication with a second inlet and a second outlet; and, optionally,
   (c) a third fluidic channel in fluidic communication with a third inlet and a third outlet,
wherein the first, second and third fluidic channels are disposed in a substantially planar substrate disposed between a first layer and a second layer; and
a chromatography plate attached to at least a portion of the substrate, wherein the first, second, and, optionally, third outlets are in fluid communication with the surface of the chromatography plate;
(B) dispensing a first solvent through the first channel of the fluidic chip such that the solvent carries the sample through the first channel, to the first outlet, and onto the chromatography plate;
(C) dispensing a second solvent through the first channel of the fluidic chip, to the first outlet, and onto the chromatography plate such that the at least one analyte in the sample is separated along a first length of the chromatography plate; and, optionally,
(D) dispensing a third solvent through the second channel of the fluidic chip, to the second outlet, and onto the chromatography plate such that the at least one analyte is concentrated along a second length of the chromatography plate.

78. A method for automatically performing thin layer chromatography comprising:
(a) engaging a fluidic cartridge comprising a sample with a cartridge interface of a system; and
(b) automatically performing with the system the steps of:
   (i) separating an analyte in the sample from contaminating materials;
   (ii) delivering the separated analyte to a surface of a thin layer chromatography plate;
   (iii) developing the plate to move the analyte into a separation zone of the plate;
   (iv) optionally concentrating the analyte with a concentration solvent; and
   (v) detecting the optionally concentrated analyte on the plate.

79. A method comprising:
(a) performing thin layer chromatography (“TLC”) on one or more analyte molecules by:
   (i) moving a liquid sample comprising the analyte molecules into a fluidic channel of a fluidic chip;
   (ii) moving the liquid sample through a phase transfer assembly comprised in a fluidic channel internal or external to the fluidic chip to exchange and/or transfer the analyte molecules from the liquid sample into a second liquid;
   (iii) delivering the second liquid from a port in the fluidic chip to a sample loading zone of a TLC plate comprising chromatography medium on a solid substrate;
   (iv) moving a separating solvent through a fluidic channel in the fluidic chip and delivering the separating solvent from the fluidic chip to the sample loading zone; and
   (v) developing the TLC plate by allowing the separating solvent to migrate with the analyte molecules into a separation zone that extends in the first dimension from the sample loading zone;
(b) optionally, concentrating the analyte by:
   (i) moving a concentrating solvent through one or more fluidic channels in the fluidic chip and delivering the concentrating solvent from the fluidic chip to one or more concentrating solvent loading zones on the TLC plate, wherein the one or more concentrating solvent loading zones are positioned in the separation zone and lateral relative to the analyte molecules; and
   (ii) further developing the TLC plate by allowing the concentrating solvent to migrate through the chromatography medium in a dimension orthogonal or oblique to the first dimension;
(c) optionally, tagging the analyte molecules with a detectable tag by:
(i) moving a fluid comprising a detectable tag through one or more fluidic channels in the fluidic chip and delivering the fluid comprising the tag to separation zone, and along the tag detect analyte molecules; and
(d) detecting, with a detector, a signal from the TLC plate indicating the presence of the analyte molecules.

80. The method of embodiment 80, wherein the liquid sample is an aqueous sample comprising an aqueous solvent and/or the second liquid is an organic solvent.

### EXAMPLES

A saliva sample is analyzed for the presence of THC by the following method:

Loading Oral Fluid and Dichloromethane (DCM) onto the Column:
1. Insert cartridge.
2. Engage the manifold 5 times.
3. Turn on the hotplate to 80° C.
4. Aspirate 600 μL of oral fluid using syringe pump 1, valve 4.
5. Aspirate 1 mL of Dichloromethane (DCM) from the reservoir bottle, using syringe pump 3, valve 3.
6 Dispense 1 mL of DCM from syringe pump 3, using valve 2.
This moves 400 ml of sample from the fluidic channel into the phase transfer assembly.
   Loading the DCM Sample from the Column onto the TLC Plate:
7. Aspirate 1 mL of DCM from the reservoir bottle, using syringe pump 3, valve 3.
8. Dispense 800 μL of DCM from syringe pump 3, using valve 2.
9. Aspirate 800 μL of DCM from the reservoir bottle, using syringe pump 3, valve 3.
10. Turn on the fan.
11. Dispense 100 μL of DCM using syringe pump 3, valve 2. Wait 5 seconds. Repeat 4 times. Next, aspirate 300 μL of DCM using syringe pump 3, valve 2. Wait 5 seconds.
A layer of cotton positioned after the silica layer contains a control, CBD. In this step, metabolites of THC, such as 11-OH-THC and 11-COOH-THC, remain on the stationary phase, while THC, CBC, and CBD move with the mobile phase into the sample loading area. The sample loading area has a length of about 2.5 cm.

Stacking THC and CBD into one straight line on the TLC plate:
12. Repeat step 11 two times.
13. Turn off the fan.
14. Aspirate 500 μL of DCM from the reservoir bottle, using syringe pump 3, valve 3.
15. Dispense 700 μL of DCM from syringe pump 3, using valve 2. Wait 15 seconds.
16. Turn on the fan.
17. Aspirate 500 μL of DCM using syringe pump 3, valve 2.
18. Change the temperature of the hot plate to 35° C. Wait 1 minute.
19. Turn off the fan and cover the TLC plate with a glass cover.
20. Aspirate 700 μL of 5 acetonitrile: 1 water (the separating solvent) from the reservoir bottle, using syringe pump 2, valve 3.

Separate CBD, CBC and THC by TLC.
21. Dispense 500 μL of 5:1 water:methanol from syringe pump 2, valve 4. Wait 40 seconds.
22. Dispense 100 μL of 5:1 from syringe pump 2, valve 4. Wait 75 seconds.
23. Dispense 100 μL of 5:1 from syringe pump 2, valve 4. Wait 80 seconds.

Concentrate THC and CBD from lines into concentrated spots:
24. Turn on the fan and turn the hotplate to 40° C. Wait 1 minute.
25. Turn off the fan.
26. Aspirate 300 μL of DCM from the reservoir bottle, using syringe pump 3, valve 3.
27. Dispense 700 μL of DCM from syringe pump 3, using valve 1.
28. Aspirate 700 μL of DCM from the reservoir bottle, using syringe pump 3, valve 3.
29. Dispense 1 mL of DCM from syringe pump 3, using valve 1.

Perform a second concentration step:
30. Aspirate 200 μL of DCM using syringe pump 3, valve 1.
31. Turn on the fan.
32. Aspirate 800 μL of DCM from the reservoir bottle, using syringe pump 3, valve 3.
33. Dispense 1 mL of DCM from syringe pump 3, using valve 1.
34. Aspirate 200 μL of DCM using syringe pump 3, valve 1.
35. Turn on the fan. Wait 30 seconds.
36. Turn off the fan.

Application of dye:
37. Dispense 300 μL of air from syringe pump 1, valve 3.
38. Aspirate 700 μL of Fast blue indicator from the reservoir bottle, using syringe pump 1, valve 5.
39. Dispense 1 mL of Fast blue indicator from syringe pump 1, using valve 6.
40. Turn on peri pump 2, to aspirate/dispense Methanol onto TLC plate. Wait 30 seconds.
41. Turn off peri pump 2.
42. Turn on fan to blow off the Fast blue indicator and Methanol from TLC plate.

Image acquisition and processing:
43. Wait 2 minutes before taking a picture of the TLC plate.
44. Do background subtraction on the image.
45. Integrate the peaks and compare the calibration standards to the unknown sample to find the unknown concentration.

As used herein, the following meanings apply unless otherwise specified. The words “can” and “may” are used in a permissive sense (i.e., meaning having the potential to), rather than the mandatory sense (i.e., meaning must). The words “include”, “including”, and “includes” and the like mean including, but not limited to. The singular forms “a,” “an,” and “the” include plural referents. Thus, for example, reference to “an element” includes a combination of two or more elements, notwithstanding use of other terms and phrases for one or more elements, such as “one or more.” The phrase “at least one” includes “one”, “one or more”, “one or a plurality”, and, therefore, contemplates the use of the term “a plurality”. The term “or” is, unless indicated otherwise, non-exclusive, i.e., encompassing both “and” and “or.” The term “any of” between a modifier and a sequence means that the modifier modifies each member of the sequence. So, for example, the phrase “at least any of 1, 2 or 3” means “at least 1, at least 2 or at least 3”. The term “about” refers to a range that is 5% plus or minus from a stated numerical value within the context of the particular usage. The term “consisting essentially of” refers to the inclusion of recited elements and other elements that do not materially affect the basic and novel characteristics of a claimed combination.

It should be understood that the description and the drawings are not intended to limit the invention to the particular form disclosed, but to the contrary, the intention is to cover all modifications, equivalents, and alternatives falling within the spirit and scope of the present invention as defined by the appended claims. Further modifications and alternative embodiments of various aspects of the invention will be apparent to those skilled in the art in view of this description. Accordingly, this description and the drawings are to be construed as illustrative only and are for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed or omitted, and certain features of the invention may be utilized independently, all as would be apparent to one skilled in the art after having the benefit of this description of the invention. Changes may be made in the elements described herein without departing from the spirit and scope of the invention as described in the following claims.

All publications, patents, and patent applications mentioned in this specification are herein incorporated by reference to the same extent as if each individual publication, patent, or patent application was specifically and individually indicated to be incorporated by reference.

What is claimed is:

1. A method comprising:
(a) performing thin layer chromatography (“TLC”) on one or more analyte molecules by:
(i) moving a liquid sample comprising the analyte molecules into a fluidic channel of a fluidic chip;
(ii) moving the liquid sample through a phase transfer assembly comprised in a fluidic channel internal or external to the fluidic chip to separate analyte molecules from contaminants;
(iii) delivering the separated analyte molecules from a port in the fluidic chip to a sample loading zone of a TLC plate comprising chromatography medium on a solid substrate;
(iv) moving a separating solvent through a fluidic channel in the fluidic chip and delivering the separating solvent from the fluidic chip to the sample loading zone; and
(v) developing the TLC plate by allowing the separating solvent to migrate with the analyte molecules into a separation zone that extends in the first dimension from the sample loading zone;
(b) optionally, concentrating the analyte by:
(i) moving a concentrating solvent through one or more fluidic channels in the fluidic chip and delivering the concentrating solvent from the fluidic chip to one or more concentrating solvent loading zones on the TLC plate, wherein the one or more concentrating solvent loading zones are positioned in the separation zone and lateral relative to the analyte molecules; and
(ii) further developing the TLC plate by allowing the concentrating solvent to migrate through the chromatography medium in a dimension orthogonal or oblique to the first dimension;
(c) optionally, tagging the analyte molecules with a detectable tag by:
(i) moving a fluid comprising a detectable tag through one or more fluidic channels in the fluidic chip and delivering the fluid comprising the tag to separation zone, and along the tag detect analyte molecules; and
(d) detecting, with a detector, a signal from the TLC plate indicating the presence of the analyte molecules.

2. The method of claim 1, wherein:
(i) the fluidic chip comprises a sample inlet port, a negative pressure port, a positive pressure port, and a liquid delivery port, fluidically connected to each other through a network of fluidic channels, wherein, a channel between the sample inlet port and the positive pressure port is intersected by a channel from the negative pressure port and a channel from the liquid delivery port, thereby defining a channel segment;
(ii) using negative pressure applied to the negative pressure port, to draw a volume of the liquid sample, sufficient to fill the channel segment; and
(iii) using positive pressure applied to the positive pressure port to push the volume through the phase transfer assembly and extract analyte from the volume.

3. The method of claim 1, wherein moving the liquid sample into the fluidic channel comprises engaging a sample container containing the sample liquid with a port in the fluidic chip.

4. The method of claim 1, wherein moving the liquid sample into the fluidic channel comprises drawing the sample through a sample port in the fluidic chip by applying negative pressure to a pressure port in the fluidic chip in communication with the sample port.

5. The method of claim 1, wherein moving liquids through fluidic channels comprises pumping the liquids with one or more pumps that apply positive pressure to the channels.

6. The method of claim 1, wherein delivering the analyte molecules to the sample loading zone comprises moving a polar, organic solvent through the fluidic channels, e.g., in an amount between about 0.1 mL and about 1 mL.

7. The method of claim 1, wherein developing the TLC plate comprises using a fan to evaporate solvent from a surface of the TLC plate.

8. The method of claim 1, wherein developing the TLC plate comprises using a temperature regulation device to heat the TLC plate.

9. The method of claim 1, wherein the sample comprises an environmental sample.

10. The method of claim 1, wherein the sample comprises a biological sample, e.g., saliva.

11. The method of claim 1, wherein the analyte comprises a psychoactive drug, e.g., THC.

12. The method of claim 1, wherein the analyte is present in the mixture at a concentration of no more than 1%.

13. The method of claim 1, wherein the analyte comprises THC and the detectable tag comprises fast blue.

14. The method of claim 1, wherein detecting comprises using a camera to detect an image from the TLC plate.

15. The method of claim 1, wherein detecting comprises quantifying an amount of detect analyte.

16. The method of claim 1, wherein detecting comprises detecting an image from the TLC plate they camera, digitizing the image and using computer software to quantify an amount of the analyte in the digitized image.

17. The method of claim 1, comprising depositing on the thin-layer chromatography plate as little as any of 50 ng, 20 ng, 5 ng, 2 ng, 1 ng or 0.2 ng of an analyte (e.g., THC) and detecting the analyte.

18. The method of claim 1, comprising passing between 200 microliters and 1000 microliters of a sample comprising an analyte in a concentration as low as any of 125 nanograms per milliliter, 50 nanograms per milliliter, 12.5 nanograms per milliliter, 2.5 nanograms per milliliter or 0.5 nanograms per milliliter, through the phase transfer assembly, and detecting the analyte.

19. The method of claim 1, performed in no more than any of 1 hour, 45 minutes, 30 minutes, 15 minutes or 5 minutes.

20. The method of claim 1, performed by a system of claim 48.

21. A cartridge for performing thin layer chromatography, comprising a fluidic chip mated to a thin layer chromatography (“TLC”) plate; wherein:
(a) the TLC plate comprises a surface comprising chromatographic medium, the surface comprising a sample loading zone, a separation zone extending in a first dimension from the sample loading zone, and, optionally, one or more concentrating solvent loading zones positioned in the separation zone; and
(b) the fluidic chip comprises:
(i) a first fluidic circuit comprising ports connected by one or more internal and, optionally, external, fluidic channels, including a sample inlet port, at least one pressure port, and a liquid delivery port; wherein the first fluidic circuit is configured to receive, by negative pressure delivered through a pressure port, a sample from the sample inlet port into a fluidic channel, and to deliver, by positive pressure delivered through a pressure port, at least part of the sample through a fluidic channel comprising a phase transfer medium, and out the liquid delivery port onto the sample loading zone; and
(ii) a second fluidic circuit comprising ports connected by one or more fluidic channels, including a separating solvent intake port and a separating solvent delivery port, wherein the separating solvent delivery port is positioned to deliver separating solvent onto the sample loading zone, wherein the second fluidic circuit optionally shares ports and/or fluidic channels with the first fluidic circuit;
(iii) optionally, one or more third fluidic circuits comprising ports connected by fluidic channels, including one or more concentrating solvent intake ports and one or more concentrating solvent delivery ports, wherein the one or more concentrating solvent delivery ports are positioned to deliver concentrating solvent to the one or more concentrating solvent loading zones;
(iv) optionally, one or more fourth fluidic circuits comprising ports connected by one or more fluidic channels, including a dye intake port and a dye delivery port, wherein the dye delivery port is positioned it to delivery dye onto the separation zone; and
(v) an aperture through the fluidic chip which exposes the separation zone.

22. The cartridge of claim 21, wherein the one or more concentrating solvent loading zones are positioned lateral relative to the sample loading zone.

23. The cartridge of claim 21, wherein the TLC plate comprises calibrants spotted on the chromatography medium.

24. The cartridge of claim 21, wherein the TLC plate comprises a solid substrate selected from glass, glass, quartz, metal, aluminum, and plastic.

25. The cartridge of claim 21, wherein the chromatography medium is selected from silica, alumina, cellulose, and polyamide.

26. The cartridge of claim 21, wherein the first fluidic circuit comprises a branched fluidic channel connected to the sample inlet port, wherein a first branch connects with the first pressure port through a branched channel, and a second branch connects with the liquid delivery port.

27. The cartridge of claim 21, wherein the fluidic channel comprising the phase transfer medium is external to the fluidic chip and connected to internal channels through ports.

28. The cartridge of claim 21, wherein the fluidic channel comprising the phase transfer medium is internal to the fluidic chip.

29. The cartridge of claim 21, wherein the phase transfer assembly comprises a filter for filtering particulate matter, a hydrophilic resin and a solid phase extraction.

30. The cartridge of claim 29, wherein the solid phase extraction medium comprises a reverse phase resin.

31. The cartridge of claim 21, wherein the second fluidic circuit is not in fluid communication with the first fluidic circuit.

32. The cartridge of claim 21, wherein the fluidic chip comprises the one or more third fluidic circuits.

33. The cartridge of claim 32, wherein the one or more third fluidic circuits comprise first and second concentrating solvent delivery ports.

34. The cartridge of claim 33, wherein the fluidic chip comprises a single third fluidic circuit.

35. The cartridge of claim 21, wherein the fluidic chip comprises the one or more fourth fluidic circuits.

36. The cartridge of claim 35, wherein the fluidic chip comprises a single fourth fluidic circuit.

37. The cartridge of claim 21, comprising one or more loading reservoirs in contact with the surface over the sample loading zone, wherein the liquid delivery port and the solvent delivery port are in fluidic communication with the one or more loading reservoirs.

38. The cartridge of claim 37, wherein the liquid delivery port and the solvent delivery port communicate with different loading reservoirs.

39. The cartridge of claim 21, comprising one or more concentrating reservoirs in contact with the surface over the one or more concentrating solvent loading zones, wherein the one or more concentrating solvent delivery ports are in fluidic communication with the one or more loading reservoirs, optionally wherein the concentrating reservoirs have an elongate shape oriented substantially parallel to the first dimension.

40. The cartridge of claim 39, wherein the concentrating reservoirs have an aspect ratio in an elongate dimension of at least 5:1 or 10:1.

41. The cartridge of claim 37 or 39, wherein one or more of the reservoirs are formed at least in part in the fluidic chip, wherein contact between the fluidic chip and the TLC plate surface forms a barrier whereby liquid in a reservoir migrates under the reservoir and through the chromatography medium.

42. The cartridge of claim 41, wherein the reservoir comprises an elongate, e.g., linear aspect that allows separation medium to travel as a substantially linear front.

43. The cartridge of claim 21, comprising a holder configured to support the fluidic chip and the TLC plate.

44. The cartridge of claim 43, wherein the holder comprises a portion configured to support the TLC chip that is recessed with respect to a portion configured to support the fluidic chip.

45. The cartridge of claim 21, further comprising a sample collector engaged with the sample inlet port.

46. The cartridge of claim 21, wherein the fluidic chip comprises mated first and second pieces, wherein one or both of the pieces comprise open channels on a face of the piece and/or apertures through the piece, wherein needing the first and second pieces closes the channels and puts to channels in communication with a plurality of the apertures.

47. The cartridge of claim 46, wherein at least one aperture through a piece communicates with an open channel on a face of the piece, or, at least one aperture through a piece communicates with an open channel on the other piece when the pieces are mated.

48. A system comprising:
(a) a cartridge interface for engaging a cartridge, wherein the cartridge interface comprises:
(i) a stage for holding the cartridge; and
(ii) a fluidics manifold comprising a plurality of fluidic ports adapted to engage ports in a cartridge when held by the holder;
(b) a fluidics assembly comprising:
(i) a plurality of containers for containing fluids;
(ii) a plurality of fluid lines; and
(iii) one or more pumps configured to pump liquids from the containers, through the fluid lines, to the fluidic ports, and to apply positive or negative pressure through at least one fluid line to at least one fluidic port;
(iv) optionally, one or more valves configured to switch fluidic connections between the containers and fluid lines leading to the fluidics manifold;
(c) a detector configured to detect a signal from the cartridge;
(d) optionally, a fan positioned to direct air to a surface of a thin layer chromatography plate of a cartridge when the cartridge is engaged with the cartridge interface.
(e) optionally, a temperature regulator positioned to regulate temperature of a cartridge engaged with the interface;
(f) a computer comprising operating software to control actions of the cartridge interface, the fluidics assembly and the detector, and, when present, the fan and the temperature regulator; and
(g) optionally, a cartridge engaged with the cartridge interface.

49. The system of claim 48, wherein the stage is slidably attached to a chassis of the system to allow placement of the cartridge on the stage when the stage is extended from the interface, and to allow engagement of the fluidics manifold when the stages retracted into the system.

50. The system of claim 48, wherein the fluidics manifold comprises at least any of three, four, five, six, seven, or eight fluidic ports.

51. The system of claim 48, wherein the plurality of containers include containers differently containing one, two, three, four, or five liquids selected from a separating solvent, an organic polar solvent, a concentrating solvent, and a dye.

52. The system of claim 51, wherein the separating solvent is selected from dichloromethane, methanol, hexanes, water, acetonitrile, dibromomethane, chloroform, toluene, acetone, methyl-thf, pentane, cyclopentyl methyl-ether, ethyl acetate, and 1-octanol.

53. The system of claim 51, wherein the organic polar solvent is selected from dichloromethane, methanol, hexanes, water, acetonitrile, dibromomethane, chloroform, toluene, acetone, methyl-thf, pentane, cyclopentyl methyl-ether, ethyl acetate, and 1-octanol.

54. The system of claim 51, wherein the concentrating solvent is selected from dichloromethane, methanol, hexanes, water, acetonitrile, dibromomethane, chloroform, toluene, acetone, methyl-thf, pentane, cyclopentyl methyl-ether, ethyl acetate, and 1-octanol.

55. The system of claim 51, wherein the dye is selected from fast blue, fast blue bb salt, fast black k, fast red TR salt, and fast garnet GBC sulfate salt.

56. The system of claim 48, wherein one or more of the pumps is a syringe pump.

57. The system of claim 48, wherein one of the pumps is a peristaltic pump.

58. The system of claim 48, wherein the one or more pumps is at least any of two, three, or four pumps.

59. The system of claim 48, wherein the fluidics assembly comprises the one or more valves.

60. The system of claim 48, wherein the detector comprises a camera.

61. The system of claim 48, wherein the detector comprises a source of light in the range of 500 nm to 600 nm.

62. The system of claim 48, comprising the fan.

63. The system of claim 48, comprising the temperature regulator.

64. The system of claim 63, wherein the temperature regulator comprises a Peltier.

65. The system of claim 48, wherein a cartridge is engaged with the cartridge interface.

66. The system of claim 65, wherein the cartridge comprises a cartridge of claim 21.

67. The system of claim 65, wherein the cartridge comprises a fluidic chip mated with a thin layer chromatography plate, wherein the chip comprises an aperture exposing a chromatography surface of the TLC plate and wherein the system comprises the detector which is configured to detect a signal from the surface.

68. A method comprising performing thin layer chromatography (“TLC”) by moving a sample from a fluidic channel in a fluidic chip onto a sample loading zone of a thin layer chromatography plate; moving a separation solvent from a fluidic channel in a fluidic chip onto the sample loading zone; and developing the TLC plate by allowing the separating solvent to separate analytes in a separation zone of the TLC plate and a first dimension.

69. The method of claim 68, further comprising, moving concentration solvent from one or more fluid channels in the fluidic chip onto one or more concentration solvent loading zones positioned in the separation zone lateral relative to the sample loading zone; and further developing the TLC plate by allowing the concentrating solvent to concentrate analytes in the separation zone in a second dimension.

70. The method of claim 68 or 69, further comprising, moving detection dye from the channel in the fluidic chip onto the separation zone and allowing the dye to tag the analytes.

71. A system comprising:
(a) a computer comprising:
(i) a processor; and
(II) a memory, coupled to the processor, the memory comprising a module comprising:
(1) computer executable instructions for implementing the method of claim 20.

72. A cartridge comprising:
(1) a fluidic chip comprising:
(a) a first fluidic channel in fluidic communication with a first inlet and a first outlet;
(b) a second fluidic channel in fluidic communication with a second inlet and a second outlet; and, optionally,
(c) a third fluidic channel in fluidic communication with a third inlet and a third outlet,
wherein the first, second and third fluidic channels are disposed in a substantially planar substrate disposed between a first layer and a second layer; and
(2) a chromatography plate attached to at least a portion of the fluidic chip, wherein the first, second, and, optionally, third outlets are in fluid communication with the surface of the chromatography plate.

73. A cartridge comprising:
(1) a fluidic chip comprising:
(a) a plurality of inlets,
(b) a plurality of outlets, and
(c) a plurality of channels in the substrate in fluid communication with at least one inlet and at least one outlet; and
(2) a thin layer chromatography plate in contact with at least a portion of the fluidic chip such at least two of the plurality of outlets are in fluid communication with the plate.

74. A cartridge comprising:
(1) a fluidic chip comprising:
(a) a plurality of reservoirs, each reservoir configured receive a liquid phase, and
(b) a plurality of channels in fluid communication with the plurality of reservoirs; and
(2) a thin layer chromatography plate in contact with at least a portion of the fluidic chip such that the plurality of reservoirs are in fluid communication with the plate.

75. A cartridge comprising:
(1) a conduit comprising at least one chromatography resin;
(2) a thin layer chromatography plate; and
(3) a fluidic chip comprising at least one channel, wherein the chromatography resin and the plate are in fluid communication through the channel.

76. A system comprising:
(A) a manifold for inserting a cartridge;
(B) a cartridge inserted in the manifold, the cartridge comprising:
(1) a fluidic chip comprising:
(a) a first fluidic channel in fluidic communication with a first inlet and a first outlet;
(b) a second fluidic channel in fluidic communication with a second inlet and a second outlet; and, optionally,
(c) a third fluidic channel in fluidic communication with a third inlet and a third outlet,
wherein the first, second and third fluidic channels are disposed in a substantially planar substrate disposed between a first layer and a second layer; and
(2) a chromatography plate attached to at least a portion of the substrate, wherein the first, second, and, optionally, third outlets are in fluid communication with the surface of the chromatography plate; and
(C) a first conduit in fluidic communication with the first inlet and a first valve, a second conduit in fluidic communication with the second inlet and a second valve, and, optionally, the third inlet.

77. A method comprising:
(A) delivering a sample comprising at least one analyte to a first inlet of a fluidic chip, wherein the fluidic chip comprises:
(a) a first fluidic channel in fluidic communication with the first inlet and a first outlet;
(b) a second fluidic channel in fluidic communication with a second inlet and a second outlet; and, optionally,
(c) a third fluidic channel in fluidic communication with a third inlet and a third outlet,
wherein the first, second and third fluidic channels are disposed in a substantially planar substrate disposed between a first layer and a second layer; and
a chromatography plate attached to at least a portion of the substrate, wherein the first, second, and, optionally, third outlets are in fluid communication with the surface of the chromatography plate;
(B) dispensing a first solvent through the first channel of the fluidic chip such that the solvent carries the sample through the first channel, to the first outlet, and onto the chromatography plate;
(C) dispensing a second solvent through the first channel of the fluidic chip, to the first outlet, and onto the chromatography plate such that the at least one analyte in the sample is separated along a first length of the chromatography plate; and, optionally,
(D) dispensing a third solvent through the second channel of the fluidic chip, to the second outlet, and onto the chromatography plate such that the at least one analyte is concentrated along a second length of the chromatography plate.

78. A method for automatically performing thin layer chromatography comprising:
(a) engaging a fluidic cartridge comprising a sample with a cartridge interface of a system; and
(b) automatically performing with the system the steps of:
(i) separating an analyte in the sample from contaminating materials;
(ii) delivering the separated analyte to a surface of a thin layer chromatography plate;
(iii) developing the plate to move the analyte into a separation zone of the plate;
(iv) optionally concentrating the analyte with a concentration solvent; and
(v) detecting the optionally concentrated analyte on the plate.

79. A method comprising:
(a) performing thin layer chromatography (“TLC”) on one or more analyte molecules by:
(i) moving a liquid sample comprising the analyte molecules into a fluidic channel of a fluidic chip;
(ii) moving the liquid sample through a phase transfer assembly comprised in a fluidic channel internal or external to the fluidic chip to exchange and/or transfer the analyte molecules from the liquid sample into a second liquid;
(iii) delivering the second liquid from a port in the fluidic chip to a sample loading zone of a TLC plate comprising chromatography medium on a solid substrate;
(iv) moving a separating solvent through a fluidic channel in the fluidic chip and delivering the separating solvent from the fluidic chip to the sample loading zone; and
(v) developing the TLC plate by allowing the separating solvent to migrate with the analyte molecules into a separation zone that extends in the first dimension from the sample loading zone;
(b) optionally, concentrating the analyte by:
(i) moving a concentrating solvent through one or more fluidic channels in the fluidic chip and delivering the concentrating solvent from the fluidic chip to one or more concentrating solvent loading zones on the TLC plate, wherein the one or more concentrating solvent loading zones are positioned in the separation zone and lateral relative to the analyte molecules; and
(ii) further developing the TLC plate by allowing the concentrating solvent to migrate through the chromatography medium in a dimension orthogonal or oblique to the first dimension;
(c) optionally, tagging the analyte molecules with a detectable tag by:
(i) moving a fluid comprising a detectable tag through one or more fluidic channels in the fluidic chip and delivering the fluid comprising the tag to separation zone, and along the tag detect analyte molecules; and
(d) detecting, with a detector, a signal from the TLC plate indicating the presence of the analyte molecules.

80. The method of claim 79, wherein the liquid sample is an aqueous sample and/or the second liquid is an organic solvent.
